(19) United States
(12) Patent Application Publication
(10) Pub. No.: US 2002/0055622 A1
(43) Pub. Date: May 9, 2002

(54) Mammalian non-erythroid Rh type C genes and glycoproteins

(76) Inventors: C.H. Huang, Flushing, NY (US); Zhi Liu, Elmhurst, NY (US)

Correspondence Address: Aglis Anilionis, Ph.D., HOFFMANN & BARON, LLP, 6900 Jericho Turnpike, Syosset, NY, 11791, US

(21) Appl. No.: 09/949,145

(22) Filed: Sep. 7, 2001

Related U.S. Application Data
(60) Provisional application No. 60/230,660, filed on Sep. 7, 2000.

(51) Int. Cl. C07H 21/02; C07H 21/04; C12Q 1/68; G01N 33/53; C07K 16/00

(52) U.S. Cl. 536/23.1; 435/6; 435/7.1; 435/320.1; 435/325; 530/389.1

(57) ABSTRACT

The present invention provides nucleic acid sequences encoding novel mammalian nonerythroid Rh type C glycoproteins, including the human homologue, RhCG and the mouse homologue, Rhcg. These Rhcg and RhCG glycoproteins have a characteristic twelve transmembrane domain structure and are predominantly expressed in kidney and testis. Also provided are recombinant vectors and host vector systems containing these nucleic acid sequences and fusion proteins incorporating fragments of Rhcg and RhCG glycoproteins. The invention further provides methods of detection of Rhcg and RhCG glycoproteins and also methods of detecting Rhcg and RhCG genes.

### RELATED APPLICATION

This application claims the benefit of U.S. Provisional Application No. 60/230,660 filed on Sep. 7, 2000.

This work was supported in part by National Institutes of Health Grant HL54459. The government may have certain rights to this invention.

### FIELD OF THE INVENTION

This invention relates to the field of molecular biology of mammalian genes and encoded proteins, and more particularly to the Rh glycoprotein gene family.

### BACKGROUND

In mammals, the Rh family includes the variable Rh polypeptides and invariant RhAG glycoprotein. These polytopic proteins are confined to the erythroid lineage and are assembled into a multisubunit complex essential for Rh antigen expression and plasma membrane integrity.

The Rh antigens were originally identified in human red blood cells. They are potent immunogens and, when incompatible, cause hemolytic disease of the newborn and blood transfusion reaction (1). They are defined by two erythroid-specific transmembrane proteins, the Rh polypeptides and the Rh-associated glycoprotein (RhAG), which form a multisubunit complex and display exodomains as D or CcEe antigens (2). The Rh polypeptides are highly polymorphic and are distinguished from RhAG by two biochemical features, i.e. palmitoylation but no glycosylation. In contrast, RhAG is largely invariant at the population level and is thought to modulate the assembly of the Rh complex and its surface expression. Human Rh polypeptides and RhAG are encoded by RHCED and RHAG loci that reside on chromosomes 1 and 6, respectively (3, 4). Despite this difference, these RH genes have originated from a common ancestor during evolution in that they are homologous in coding sequence and are grossly similar in exon/intron organization (5, 6).

Rh proteins (and their complex) are necessary for the maintenance of red blood cell morphology and plasma membrane integrity. The Rh deficiency syndrome, a rare inherited form of hemolytic anemia, is caused by mutations at the RHAG or RHCED locus (2). In this disorder, red blood cells deficient in all Rh antigens exhibit spherostomatocytosis and multiple membrane abnormalities (7), implying that Rh proteins have some functional roles in membrane physiological processes. At the level of secondary structure, RhAG and RhCE/D bear a 12-transmembrane topology resembling many transporters (8) and thus are likely to possess a transporter activity across the lipid bilayer. Ammonium or amino group-containing compounds have been suggested as candidate ligands for erythroid Rh proteins, owing to their connective sequence similarity with the NH₄⁺ transporters from bacteria, yeast, and plants (2, 9).

The Rh family of genes and proteins is rooted deeply in evolution, and the erythroid homologues of human RHAG and RHCED genes are coexpressed in red blood cells of all mammalian species. The closest RHAG and RHCED relatives are those from nonhuman primates, as they share not only high sequence identity but also some antigen reactivity (10, 11). The mouse erythroid Rhag and Rhced are identical to their human counterparts in the arrangement of exon/intron junctions, in the conservation of chromosome synteny, and in the pattern of coexpression (12). Furthermore, Rh homologues have been identified in primitive organisms, e.g. the unicellular slime mold Dictyostelium discoideum (2), marine sponge Geodia cydonium (13), and earthworm Caenorhabditis elegans (14, 15). Structurally, these primitive Rh forms are more similar to RhAG than they are to RhCE/D, providing some important insights into the origin of erythroid members from nonerythroid ancestors.

ABBREVIATIONS: RBC, red blood cell; TM, transmembrane; GSP, gene-specific primer; RACE, rapid amplification of cDNA ends; PCR, polymerase chain reaction; GFP, green fluorescence protein; BAC, bacterial artificial chromosome; PAGE, polyacrylamide gel electrophoresis; nt, nucleotide; bp, base pair(s); kb, kilobase(s); UTR, untranslated region; CPMM, canine pancreatic microsomal membranes.

### SUMMARY OF THE INVENTION

The present invention provides isolated nucleic acid molecules comprising a nucleotide sequence having at least 60% sequence identity with at least a portion of the human Rh type C gene, RhCG or at least a portion of the mouse Rh type C gene, Rhcg.

Also provided are isolated nucleic acids which hybridize under stringent conditions with a nucleic acid having the nucleotide sequence of RhCG, or Rhcg, or a sequence complementary to RhCG, or Rhcg.

Further the invention provides isolated nucleic acids comprising a nucleotide sequence having at least 60% sequence identity with at least a portion of the human Rh type C gene, RhCG or at least a portion of the mouse Rh type C gene, Rhcg wherein the nucleotide sequence encodes a protein comprising an amino acid sequence at least 60% identical to at least a portion of the human Rh type C glycoprotein or to at least a portion of the mouse Rh type C glycoprotein.

Further yet, the invention provides the above-identified isolated nucleic acids encoding a protein having RhCG glycoprotein or Rhcg glycoprotein activity. In particular embodiments the RhCG glycoprotein or Rhcg glycoprotein activity is NH₄⁺ ion transporter activity.

Also further provided are fragments of a nucleic acid molecule comprising a nucleotide sequence having at least 60% sequence identity with at least a portion of the human Rh type C gene, RhCG or a portion of the mouse Rh type C gene, Rhcg encoding a protein having RhCG or Rhcg activity; or encoding an epitope of RhCG or Rhcg.

Yet further, in one aspect, the invention provides recombinant vectors comprising a nucleic acid molecule comprising a nucleotide sequence having at least 60% sequence identity with at least a portion of the human Rh type C gene, RhCG or a portion of the mouse Rh type C gene, Rhcg. In certain embodiments the vector may encode a protein having an amino acid sequence at least 60% identical to at least a portion of the RhCG glycoprotein or a portion of Rhcg glycoprotein. The vector may be transformed into a suitable host cell.

In another aspect, the invention provides isolated a protein or peptide comprising an amino acid sequence at least 60% identical to at least a portion of the RhCG glycoprotein or a portion of Rhcg glycoprotein.

Also provided, in another aspect, is a fusion protein comprising a fragment of the protein or peptide comprising an amino acid sequence at least 60% identical to at least a portion of the RhCG glycoprotein or a portion of Rhcg glycoprotein.

Also provided, in yet another aspect, is an antibody that specifically binds to an epitope of RhCG glycoprotein or to an epitope of Rhcg glycoprotein.

In yet another aspect, the invention provides gene specific probes that are specific for Rh glycoprotein genes.

Further, in yet another aspect, the invention provides an isolated nucleic acid molecule comprising a functional RhCG regulatory region or a functional Rhcg regulatory region.

Further, the invention provides a hybrid gene under Rh type C gene regulation comprising an upstream nucleic acid regulatory sequence of the RhCG gene and a coding sequence of a gene to be regulated.

In a further aspect the invention provides a method of detecting an Rhcg or an RhCG glycoprotein in a sample, said method comprising: providing a sample to be tested, contacting the sample with an antibody that specifically binds to an epitope of RhCG glycoprotein or an Rhcg glycoprotein under conditions suitable for binding, assessing the specific binding to the antibody, and thereby detecting the presence of an epitope of Rhcg or RhCG glycoprotein in the sample.

In yet a further aspect the invention provides a method of detecting an Rhcg or RhCG nucleotide sequence in a sample, said method comprising: providing a nucleic acid sample, contacting the sample with a nucleic acid probe that hybridizes with the human Rh type C gene, RhCG or the mouse Rh type C gene, Rhcg, to form a mixture, incubating the mixture under conditions suitable for hybridization, detecting the nucleic acid probe hybridized to the sample, and thereby detecting the presence of an RhCG or Rhcg nucleotide sequence in the sample.

### BRIEF DESCRIPTION OF THE FIGURES

FIG. 1. The complete cDNA and predicted amino acid sequences of human RhCG (SEQ ID No.: 1 AF193809) and mouse Rhcg (SEQ ID No.: 2 AF 193810) proteins.

The sequences are aligned using the Clustal W program. Amino acid changes of similar nature are shown in boldface, and those of different nature are circled. Deletions are denoted by dashes. The N-glycosylation motifs in RhCG are overlined, and those in Rhcg are underlined. Nucleotide and amino acid numbers are counted by reference to the first position of ATG codon as 11. The polyadenylation signal AATAAA in the 3′-UTR of the two genes is underlined. (RhCG Open reading frame is SEQ ID No.: 3; Rhcg Open reading frame is SEQ ID No.: 4; RhCG cDNA is SEQ ID No.: 5, and Rhcg cDNA is SEQ ID No.: 6.

FIG. 2. The membrane topology and charge distribution of RhCG.

Human RhCG and mouse Rhcg show a nearly identical hydropathy profile as calculated in Kyte-Doolittle scale. For brevity, only the model for RhCG is shown. The 12-TM helices and their amino acid positions defining the boundary are indicated. Different circles denote groups of amino acids: solid circles, hydrophobic Phe, Ile, Leu, Met, Val, and Trp; gray circles, Gly, Ala, and Pro; open circles, polar Ser, Cys, Thr, Asn, Gln, and Tyr; +, positively charged Lys, Arg, and His; and −negatively charged Asp and Glu. The only N-glycosylation site present in the first exo loop is illustrated.

FIG. 3. Percentage of identity and phylogenetic relationship between RhCG/Rhcg and other homologs.

Upper panel, percentage of identity of protein sequences of the various Rh homologs obtained by MegAlign. Boxed area shows the percentage of identity of the human and mouse orthologous pairs. Lower panel, phylogenetic tree of the Rh family of proteins. Organisms are as follows: Hs, Homo sapiens; Mm, Mus musculus (house mouse); Dr, Danio rerio (zebrafish); Gc, G. cydonium (marine sponge); Ce, C. elegans (nematode); Dm, Drosophila melanogaster (fruit fly); and Dd, D. discoideum (slime mold). Note that both RhCG/Rhcg and RhBG/Rhbg belong to the nonerythroid group, as they merge at the same branch point. The GenBank™ accession numbers are as follows: Hs.RHBG, AF193807; Mm.Rhbg, AF193808; Hs.RHCG, AF193809; Mm.Rhcg, AF193810; Hs.RHAG, AF031548; Mm.Rhag, AF057526; Hs.RHD, L08429; Hs.RHCE, X54534; Mm.Rhced, AF547524; Dr.Rhg, AF209468; Gc.Rhg, Y12397; Ce.Rhp-1, AF183390; Ce.Rhp-2, AF183391; Dm.Rhp, AF193812; and Dd.RhgA, AF193811.

FIG. 4. Chromosomal location of human RHCG and mouse Rhcg.

A, diagram for the location of RHCG at 15q25. DNAs purified from RHCG BAC clones 300M8 and 2160119 were used as fluorescence in situ hybridization probes and gave the same result. B, Rhcg map on chromosome 7 (the centromere toward the top). A 3-cM scale bar is indicated. Loci mapping to the same position are listed in alphabetical order. Corresponding human map positions for underlined loci are listed to the left of the chromosome bar.

FIG. 5. Organization of human RHCG and mouse Rhcg genes and comparison with the erythroid RHAG and Rhag genes.

The size of exons (E1-E11) is counted in base pairs, and that of introns is counted in kilobase pairs. The noncoding exon for the 3′-UTR downstream of the stop codon in each gene is shaded. The human RHCG GenBank™ accession numbers are AF219981-AF219986. The human RHAG GenBank™ accession numbers are AF238372-AF238377.

FIG. 6. Assignment of splice sites and exon/intron junctions between RHCG and Rhcg.

Exons are in uppercase letters, and interval sequences are omitted. Acceptor and donor splice sites are in lowercase letters. Amino acids encoded by exon borders are listed. Stars denote the stop codon and polyadenylation signal. Note that exon 11 encodes no amino acid sequence.

FIG. 7. Nucleotide sequences of the 5′ region of human RHCG (SEQ ID No.: 7) and mouse Rhcg genes (SEQ ID No.: 8).

A, RHCG; B, Rhcg. Potential cis-acting motifs known to bind to the various transcription factors are boxed, and their orientation is indicated above (sense) or below (antisense) the sequences. CpG dinucleotides are clustered in the region and are shown in boldface. The major transcription, start site (−24A in RHCG and −122A in Rhcg) is denoted by a bent arrow. The first position of ATG initiation codon is numbered 11. The partial amino acid sequence of exon 1 is shown.

FIG. 8. Northern blot analysis.

A, RHCG expression in human adult and fetal tissues. B, Rhcg expression in mouse adult tissues. The size marker is indicated. Tissues are denoted above each panel. s. muscle, skeletal muscle; s. intestine, small intestine. Each lane was loaded with 2 mg of poly(A)⁺ RNAs. The 2.0-kb major band is indicated with an arrow. The actin cDNA hybridization with these blots was relatively uniform.

FIG. 9. Rhcg expression in adult mouse testis and kidney.

A, in situ RNA hybridization with a sense control probe. B, strong Rhcg expression is detected in adult mouse testis. C, high magnification of the boxed area in B showing expression in the seminiferous tubules (indicated by arrow). D, strong Rhcg expression was detected in mouse adult kidney. E and F, high magnifications showing Rhcg expression in the kidney collecting tubules (arrowheads). A-E, dark field microphotographs; F. bright field micrograph.

FIG. 10. Localization of RhCG to the plasma membrane by confocal microscopy.

Cultured HEK293 or HeLa cells were transfected with either RHCG-GFP (cloned in pEGFP-N3) or GFP-RHCG fusion plasmid (cloned in pEGFP-C1). Images were collected on a Bio-Rad MRC confocal laser scanning microscope. The panels are designated as follows: A-C, homologous HEK293 cells; D-F, heterologous HeLa cells; A and D, positive controls (pEGFP-N3 vector alone); B and E, GFP-RHCG fusion construct; C and F, RHCG-GFP fusion construct.

FIG. 11. In vitro transcription-coupled translation and N-glycosylation analysis of RhCG protein.

A, analysis of in vitro translation products by 12% reducing SDS-PAGE in the absence of CPMM. Lanes 1-4 are positive and negative controls: lane 1, yeast a-mating factor; lane 2, luciferase; lane 3, DNA omitted; lane 4, pYES2 vector. Lane 5, pYES2/ RhCG. Lane 6, pcDNA3.1/RhCG-Myc tag. B, analysis of in vitro translation products by reducing 12% PAGE after incubation with CPMM. Lane 1, yeast a-mating factor; lane 2, DNA omitted; lane 3, pYES/RhCG; lane 4, pcDNA3.1/RhCG-Myc tag. Note the up-shift of RhCG in B. Molecular mass markers (in kDa) are indicated at the left margin.

FIG. 12. Western blot analysis and N-glycanase treatment of membrane RhCG.

Plasma membrane proteins were prepared from stable HEK293 cells, fractionated by 12% SDS-PAGE, and blotted onto Hybond NC membranes. RhCG was visualized either by RhCG tail-specific antisera (A) or by anti-Myc monoclonal antibody (B). Dilution of the two antibodies is shown. Human red blood cell membranes were used as controls (lanes 1 and 2). 1 and — indicate the presence and absence, respectively, of reducing agent dithiothreitol (DTT) and N-glycanase PNGase. HRP, horseradish peroxidase. Molecular mass markers are shown at the left margin.

### DETAILED DESCRIPTION OF THE INVENTION

DEFINITIONS

“Isolated proteins” When a protein is isolated, this means that it is essentially free of other proteins. Essentially free from other proteins means that it is at least 90%, preferably at least 95% and, more preferably, at least 98% free of other proteins. Isolated proteins may be recognized as single bands after electrophoresis in SDS acrylamide gels (25) and staining with coomassie blue or silver stain.

“Essentially Pure” A protein or nucleic acid is essentially pure, when the protein or nucleic acid is free not only of other proteins and nucleic acids, but also of other materials used in its isolation and identification, such as, for example, sodium dodecyl sulfate and other detergents. The protein or nucleic acid is at least 95% free, preferably at least 98% free and, more preferably, at least 99% free of such materials.

An “oligonucleotide” or “oligomer” is a stretch of nucleotide residues which has a sufficient number of bases to be used in a polymerase chain reaction (PCR). These short sequences are based on (or designed from) genomic or cDNA sequences and are used to amplify, confirm, or reveal the presence of an identical, similar or complementary DNA or RNA in a particular cell or tissue. Oligonucleotides or oligomers comprise portions of any nucleic acid, preferably DNA sequence having at least about 10 nucleotides and as many as about 100 nucleotides, preferably about 15 to 50 nucleotides. They may be chemically synthesized and may be used as probes.

“Probes” are nucleic acid sequences of variable length, preferably between at least about 10 and as many as about 3,000 nucleotides, depending on use. They are used in the detection of identical, similar, or complementary nucleic acid sequences. Longer length probes are usually obtained from a natural or recombinant source, are highly specific and much slower to hybridize than oligomers. They may be single- or double-stranded and carefully designed to have specificity in PCR, hybridization membrane-based, or ELISA-like technologies.

“Reporter” molecules are chemical moieties used for labelling a nucleic or amino acid sequence. They include, but are not limited to, radionuclides, enzymes, fluorescent, chemi-luminescent, or chromogenic agents. Reporter molecules associate with, establish the presence of, and may allow quantification of a particular nucleic or amino acid sequence.

A “portion” or “fragment” of a polynucleotide or nucleic acid comprises all or any part of the nucleotide sequence having fewer nucleotides than about 3 kb, preferably fewer than about 1 kb which can be used as a probe. Such probes may be labelled with reporter molecules using nick translation, Klenow fill-in reaction, PCR or other methods well known in the art. After pretesting to optimize reaction conditions and to eliminate false positives, nucleic acid probes may be used in Southern, northern or in situ hybridizations to determine whether DNA or RNA encoding the RhCG or Rhcg protein is present in a biological sample, cell type, tissue, organ or organism.

“Control elements” or “regulatory sequences” are those nontranslated regions of the gene or DNA such as enhancers, promoters, introns and 3′ untranslated regions which interact with cellular proteins to carry out replication, transcription, and translation. They may occur as boundary sequences or within the coding region of the gene. They function at the molecular level and along with hormones and other biological messenger molecules as well as regulatory genes, the regulatory sequences are important in the control of development, growth, differentiation and aging processes.

“Chimeric” molecules are polynucleotides which are created by combining one or more of nucleotide sequences of this invention (or their parts) with additional nucleic acid sequence(s). Such combined sequences may be introduced into an appropriate vector and expressed to give rise to a chimeric polypeptide which may be expected to be different from the native molecule in one or more of the following ion transporter characteristics: cellular location, distribution, ligand-binding affinities, interchain affinities, degradation/turnover rate, signalling, etc.

“Active” is that state which is capable of being useful or of carrying out some role. It specifically refers to those forms, fragments, or domains of an amino acid sequence which display the biologic and/or immunogenic activity characteristic of the naturally occurring RhCG or Rhcg.

“Naturally occurring RhCG or Rhcg” refers to a polypeptide produced by cells which have not been genetically engineered or which have been genetically engineered to produce the same sequence as that naturally produced. Specifically contemplated are various polypeptides which arise from post-translational modifications. Such modifications of the polypeptide include but are not limited to acetylation, carboxylation, glycosylation, phosphorylation, lipidation and acylation.

A “signal or leader sequence” is a short amino acid sequence which or can be used, when desired, to direct the polypeptide through a membrane of a cell. Such a sequence may be naturally present on the polypeptides of the present invention or provided from heterologous sources by recombinant DNA techniques.

An “oligopeptide” is a short stretch of amino acid residues and may be expressed from an oligonucleotide. It may be functionally equivalent to and either the same length as or considerably shorter than a “fragment,” “portion,” or “segment” of a polypeptide. Such functional binding sequences, such as epitopes comprise a stretch of amino acid residues of at least about 5 amino acids and often about 17 or more amino acids, typically at least about 9to 13 amino acids, and of sufficient length to display biologic and/or immunogenic activity. Functional biologic activity, such as ion transporter activity may reside in a domain of at least about 25 amino acids, more typically about 50-100 amino acids and in some cases may require at least about 100-200 amino acids to preserve function.

A “mammal” as used herein may be defined to include human, domestic (cats, dogs, etc), agricultural (cows, horses, sheep, goats, chicken, etc) or test species (mice, rats, rabbits, simians, etc).

A “homologue” of the RHCG or Rhcg glycoprotein or of the unglycosylated form of the protein can be, for example, a substitution, addition, or deletion mutant of the protein. For example, it is preferred to substitute amino acids in a sequence with similar or equivalent amino acids. Groups of amino acids known normally to be equivalent are:

(a) Ala(A), Ser(S), Thr(T), Pro(P), Gly(G);

(b) Asn(N), Asp(D), Glu(E), Gln(Q);

(c) His(H), Arg(R), Lys(K);

(d) Met(M), Leu(L), Ile(I), Val(V); and

(e) Phe(F), Tyr(Y), Trp(W).

“Substitutions” are conservative in nature when the substituted amino acid has similar structural and/or chemical properties. Examples of conservative replacements are substitution of a leucine with an isoleucine or valine, an aspartate with a glutamate, or a threonine with a serine.

Amino acid “insertions” or “deletions” are changes to or within an amino acid sequence. They typically fall in the range of about 1 to 10 amino acids in length. The variation allowed in a particular amino acid sequence may be experimentally determined by producing the peptide synthetically or by systematically making insertions, deletions, or substitutions of nucleotides in the RhCG or Rhcg sequence using recombinant DNA techniques.

“Percent identity” is calculated as the percent contiguous amino acids or nucleotide bases that are identical in the two sequences compared. Substitutions, additions, and/or deletions in an amino acid or nucleotide sequence can be made. The sequences to be compared are aligned to give the maximum number of identities of side by side amino acids or nucleotides, with the lowest number of unmatched gaps or loops. Often this may be achieved by aligning the two sequences by eye. If visual inspection is insufficient, nucleic acid molecules may be aligned in accordance with the methods described in George, D. G. et al., in Macromolecular Sequencing and Synthesis, Selected Methods and Applications, p127-149, Alan R. Liss, Inc. (1998), see formula 4 at page 137 using a match score of 1, a mismatch score of 0 and a gap penalty of −1.

Preferably the protein encoded by the nucleic acid of the invention continues to satisfy the functional criteria described herein. An amino acid or a nucleotide sequence that is substantially the same as another sequence, but that differs from the other sequence by means of one or more substitutions, additions, and/or deletions, is considered to be a similar or equivalent sequence. Preferably, less than 40%, more preferably less than 30%, and still more preferably less than 20%, and optimally less than 10% of the total number of amino acid residues or nucleotides in a sequence are substituted, added, or deleted from the protein or the nucleic acid of the invention.

“Percent sequence similarity” in a comparison of two aligned amino acid sequences as used herein means the percent of conserved or similar amino acid residues in a stretch of amino acids optimally aligned as described above.

“Homologues and fragments of RhCG and Rhcg” The invention includes homologues and fragments of RhCG and Rhcg proteins as defined above, and also includes recombinant nucleotide variants, and fragments of nucleic acids, whether DNA, including genomic DNA or cDNA, or RNA including spliced or unspliced messenger RNA, that encode RhCG or Rhcg.

High stringency conditions are defined in a number of ways. In one definition, stringent conditions are selected to be about 25° C. lower than the thermal melting point (Tₘ) for DNA or RNA hybrids longer than 70 bases, and 5° C. lower than the Tₘ for shorter oligonucleotides (11-70 bases long). The Tₘ is the temperature (under defined ionic strength and pH, e.g. 6XSSC at 65 deg.C.) at which 50% of the target sequence hybridizes to a perfectly matched sequence. Typical stringent conditions are those in which the salt concentration is about 0.02 M at about pH 7 and the temperature is calculated as described below.

The following equations are used to calculate the Tₘ of the following hybrids: For DNA hybrids of more than 70 nucleotides:

Tₘ=81.5° C.+16.6 log[M⁺]+41(%G+C)−0.63(% formamide)−(600/L).

For DNA: RNA hybrids of more than 70 nucleotides:

Tₘ=79.8° C.+18.5 log[M⁺]+0.584(%G+C)+0.118(%G+C)²−0.5(% formamide)−820/L.

For DNA or RNA hybrids of 14-70 bases:

Tₘ=81.5° C.+16.6log[M⁺]+0.41(%G+C)−600/L.

Where

Tₘ=thermal melting temperature;

%G+C =percentage of total guanine and cytosine bases in the DNA, expressed as a mole fraction;

[M⁺]=log of the monovalent cation concentration, usually sodium, expressed in molarity in the range of 0.01 M to 0.4 M; and

L=length of the hybrid in base pairs;

%A+T=percentage of total adenine and thymine bases in the DNA and expressed as a mole fraction.

The Invention

This invention provides nucleic acids and proteins related to a class of mammalian non-erythroid glycoproteins called Rh type C glycoproteins. The human and the mouse counterparts are RhCG and Rhcg, respectively.

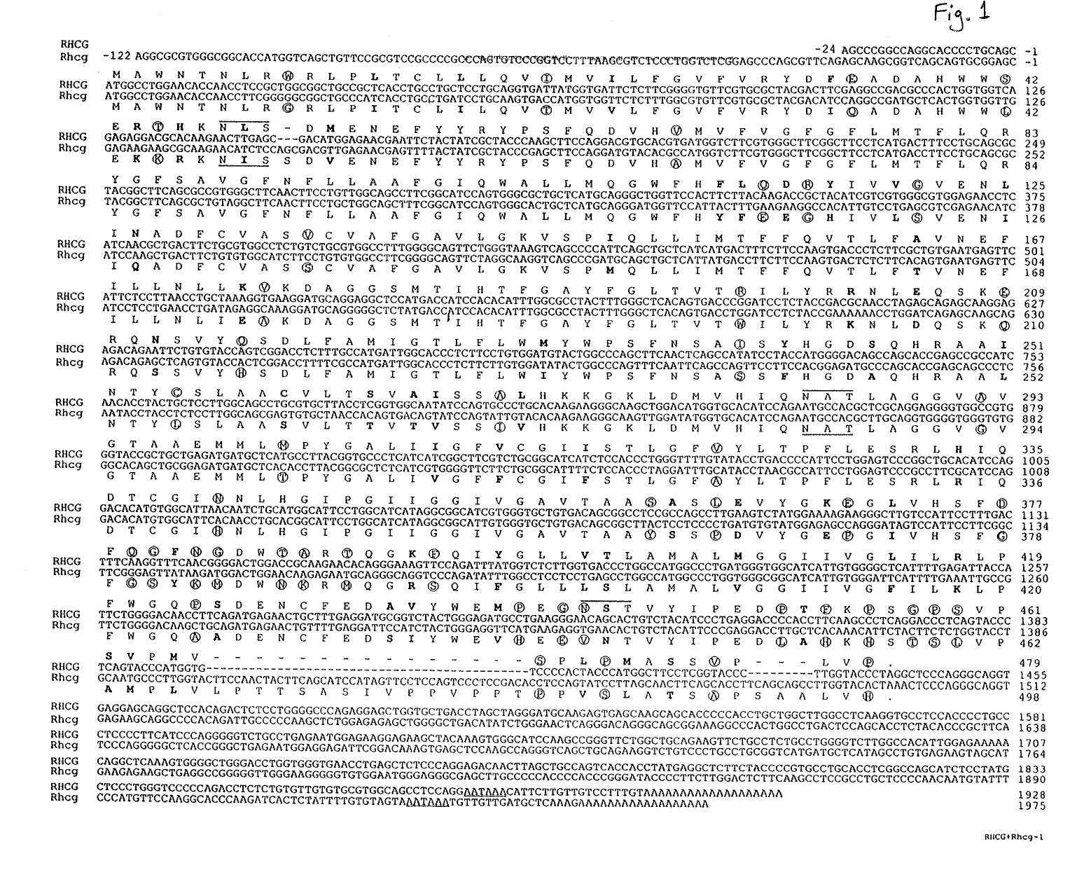

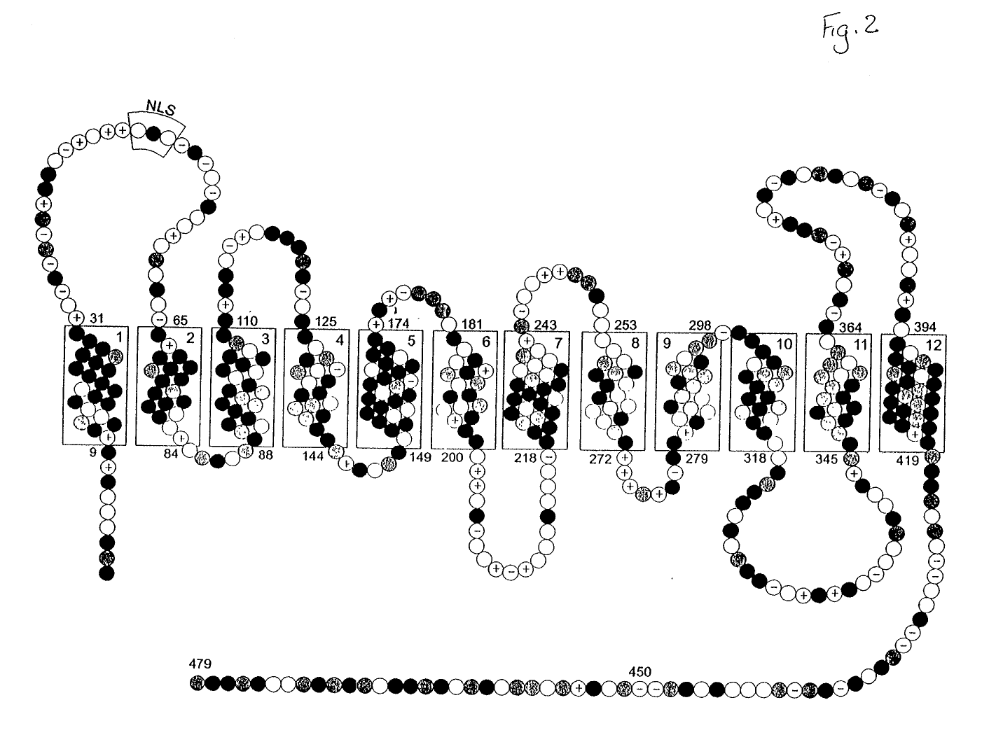

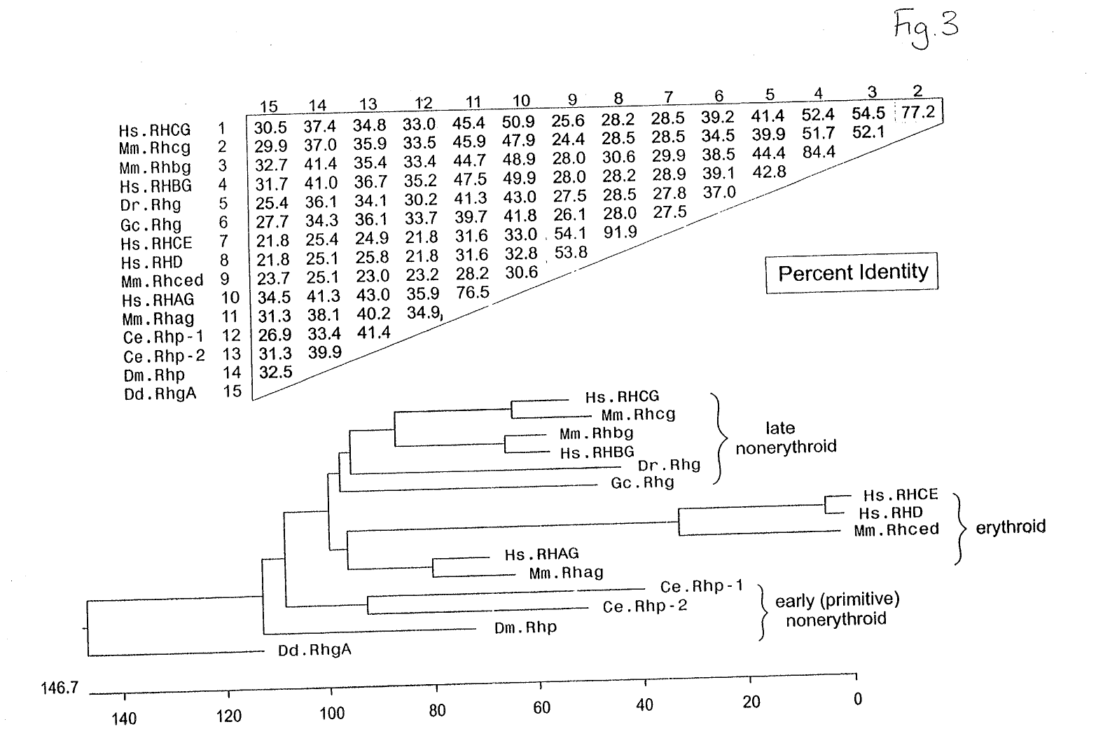

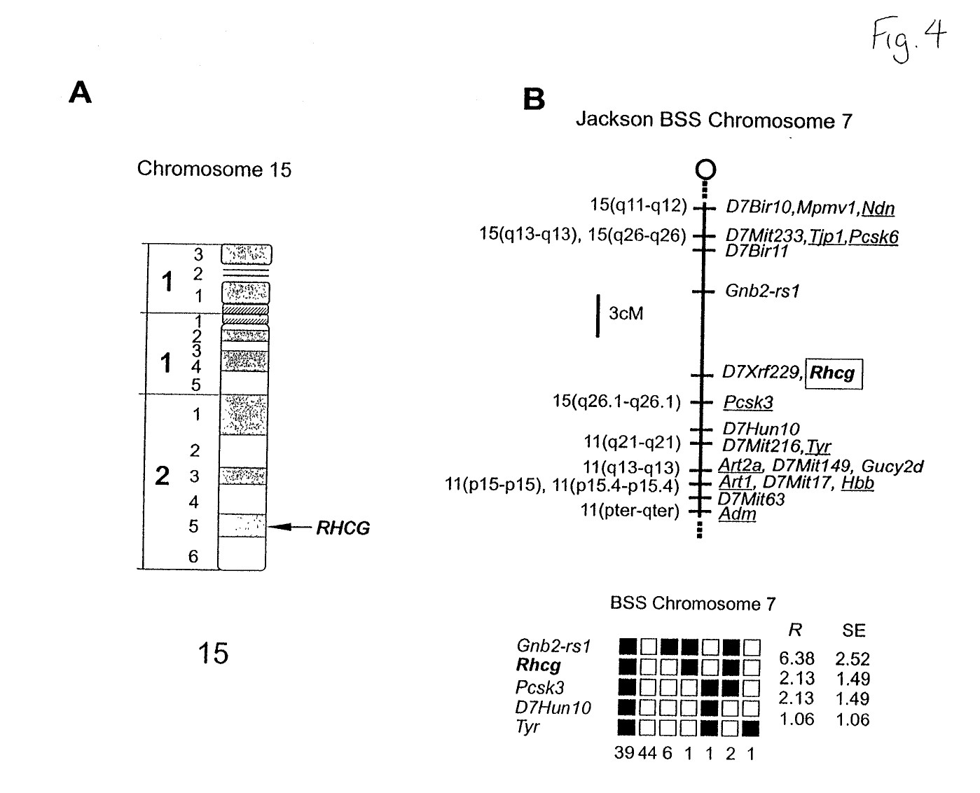

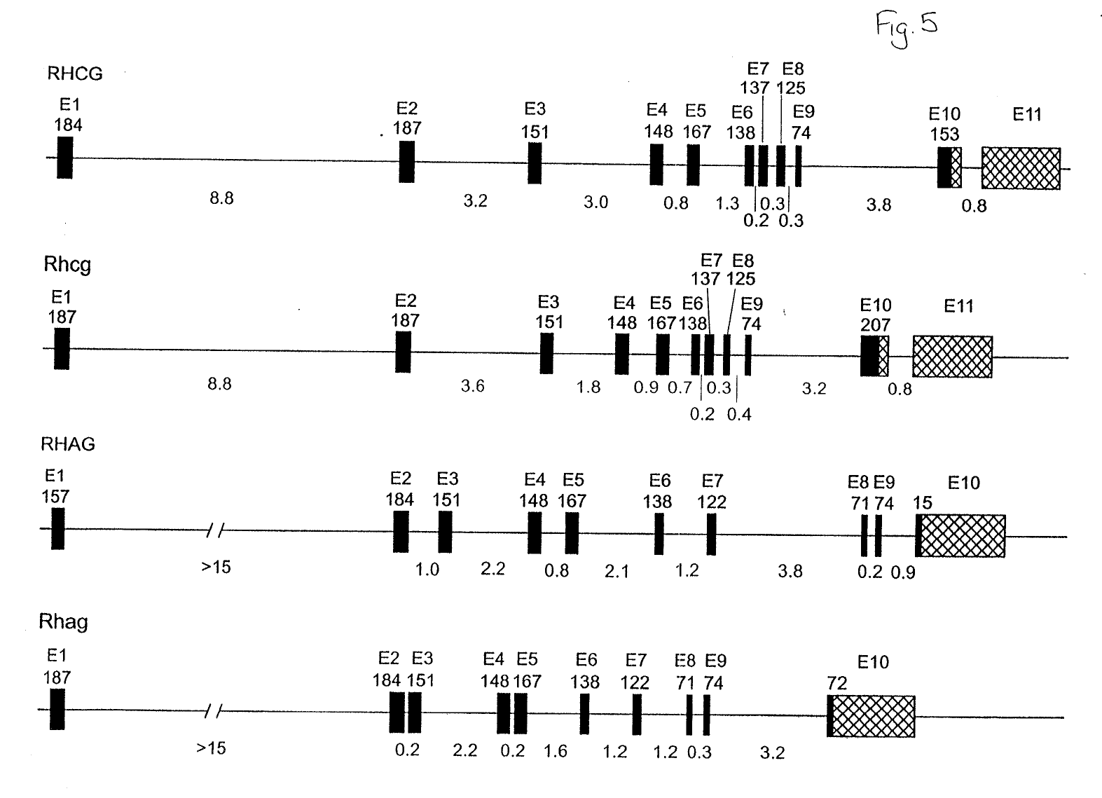

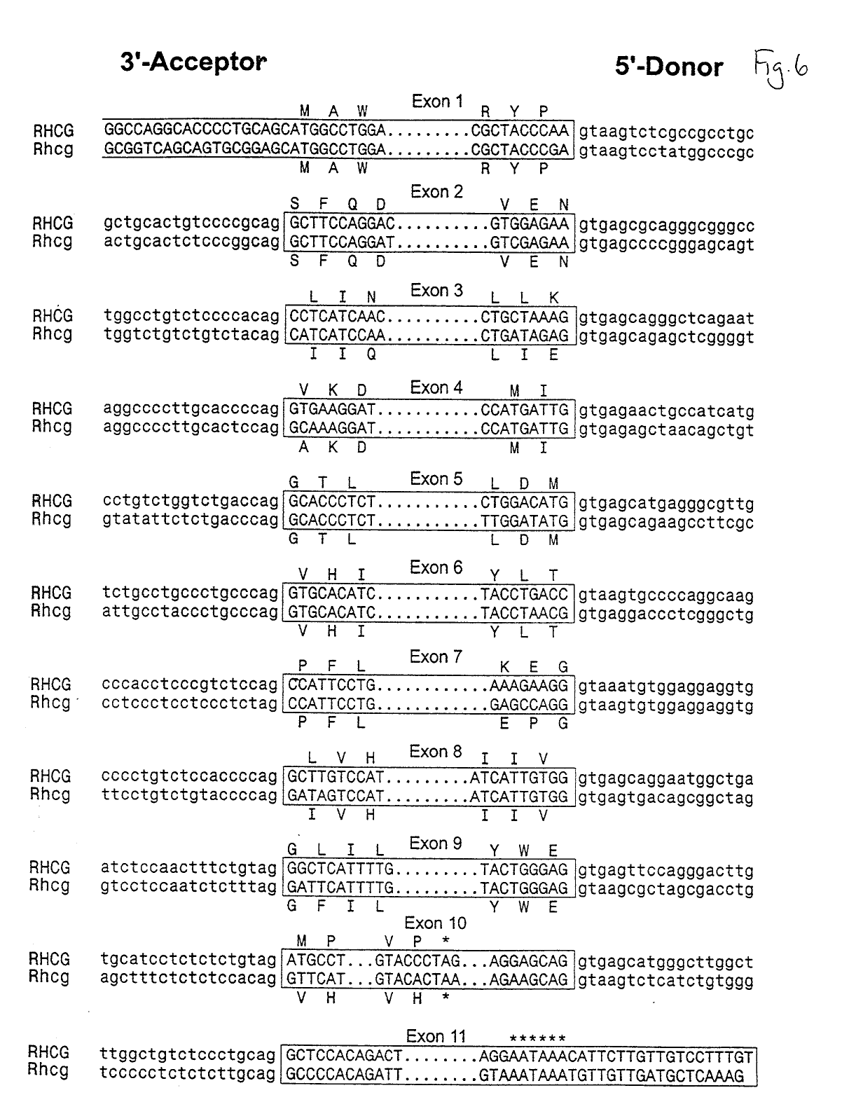

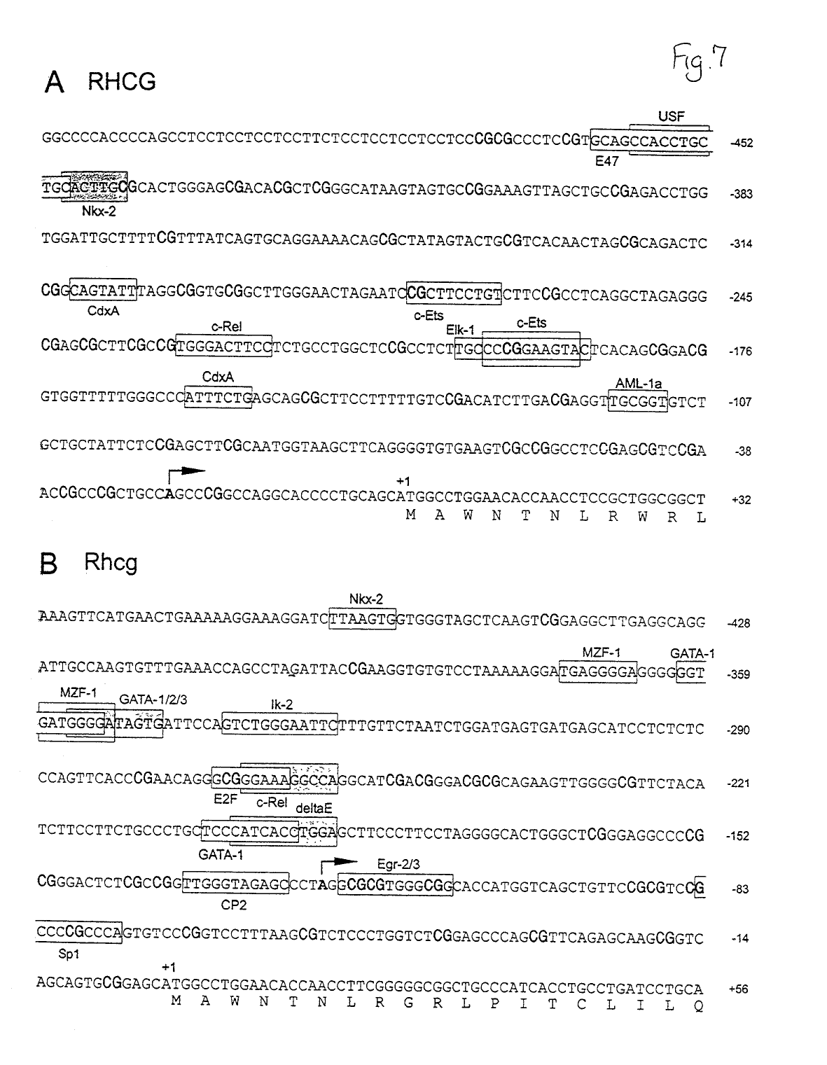

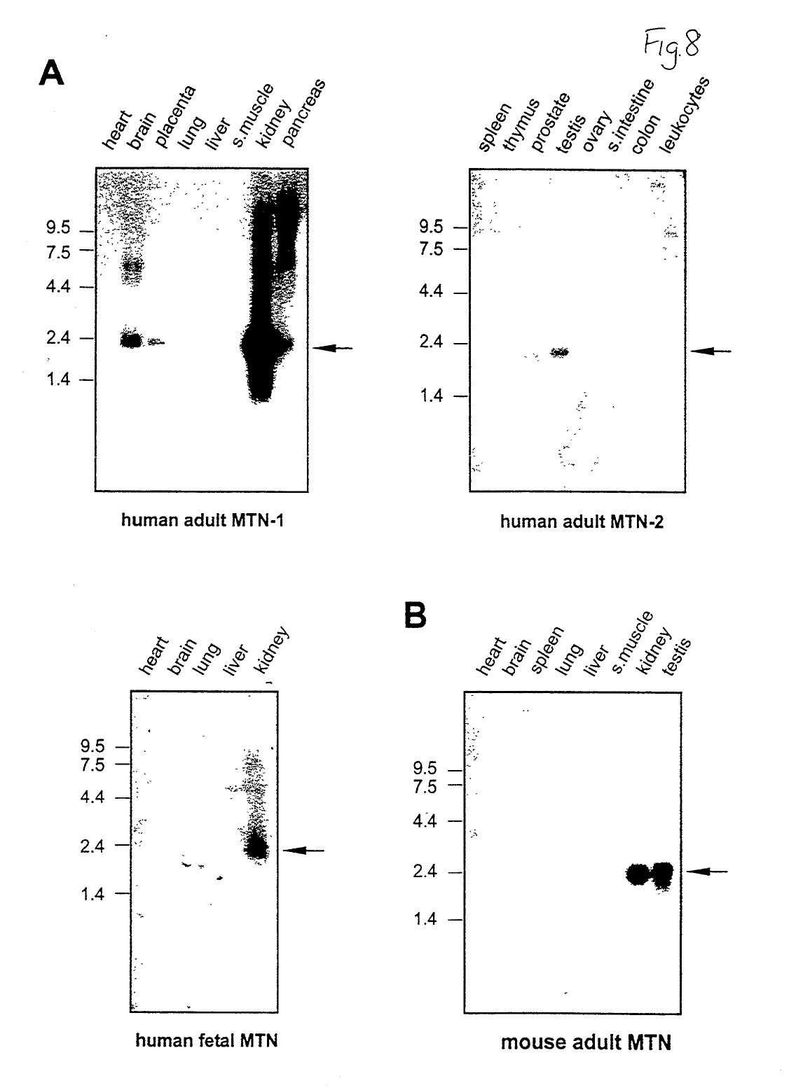

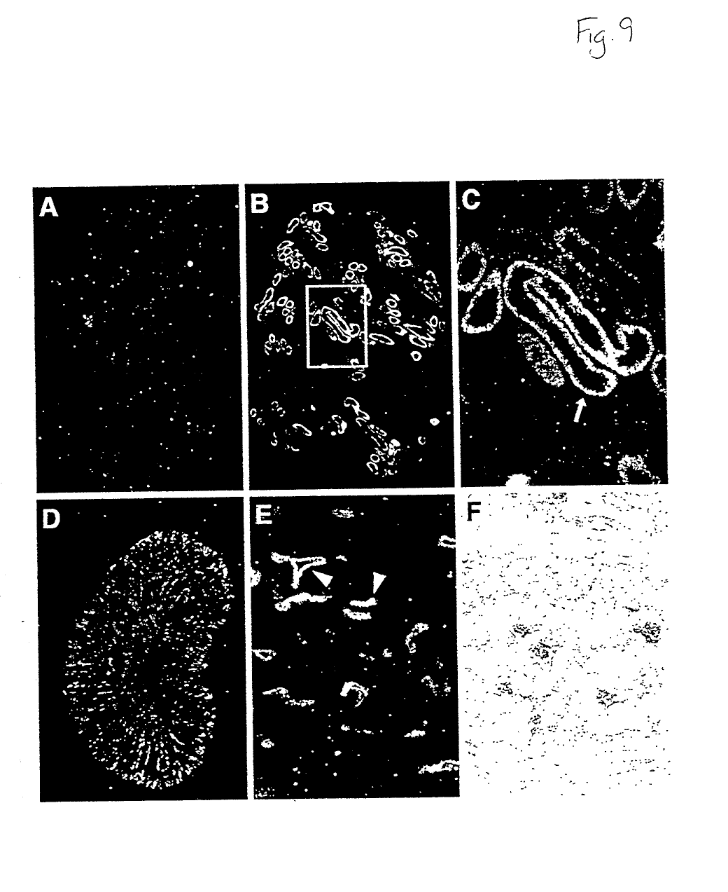

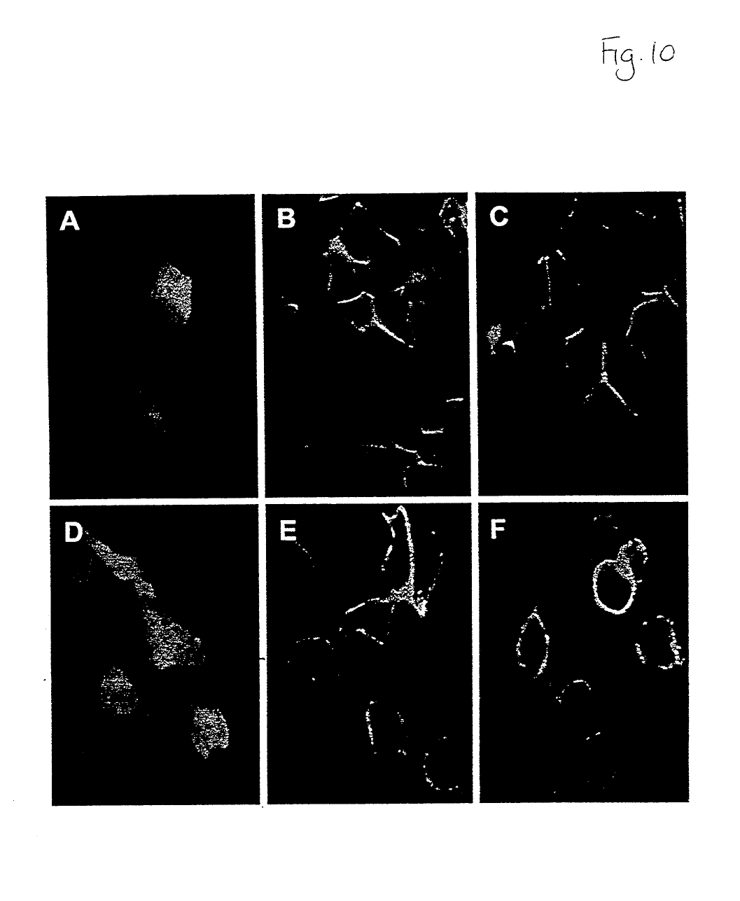

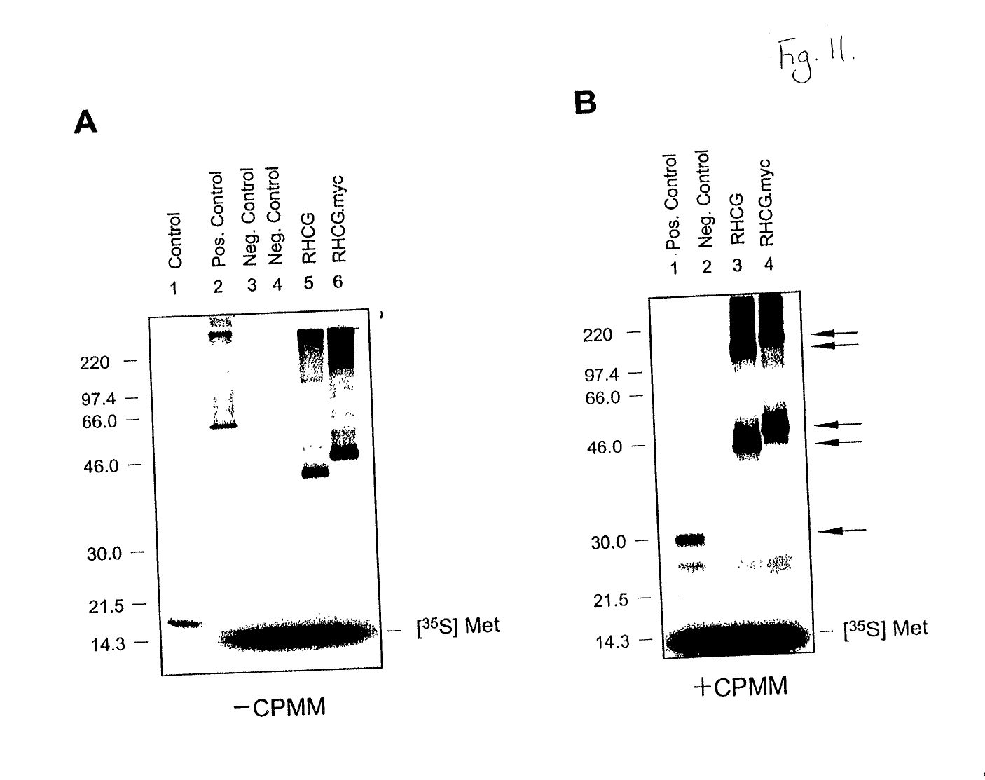

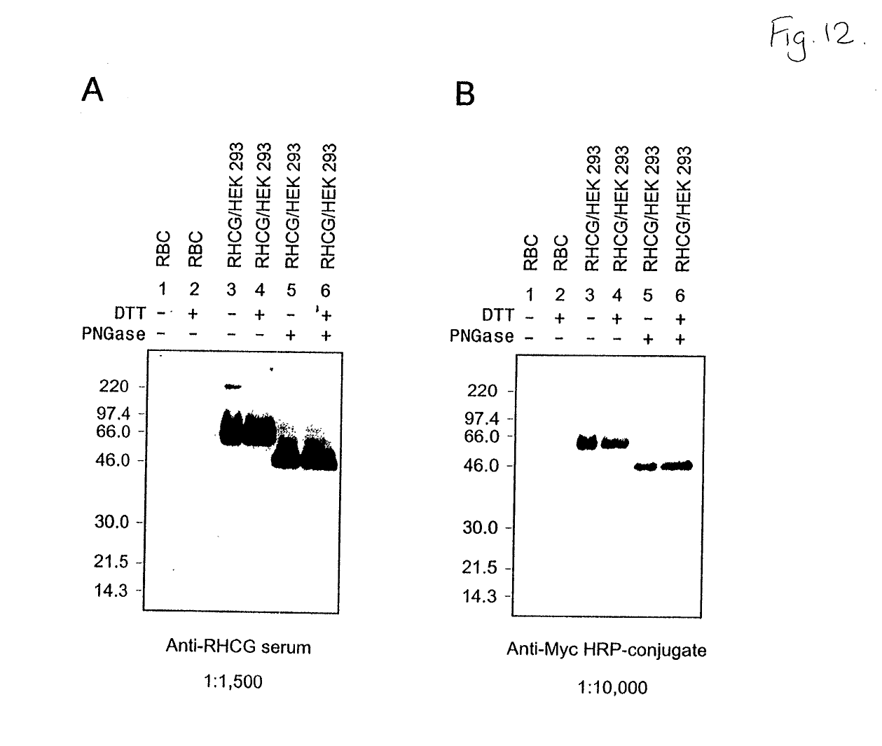

In a first embodiment the isolated nucleic acid molecules of the present invention comprise an homologous nucleotide sequence having at least about 60% sequence identity with at least a portion of the RhCG sequence (SEQ ID No.: 1) or the Rhcg sequence (SEQ ID No.: 2) shown in FIG. 1. The nucleic acid sequence is preferably greater than about 15 bases long, more preferably greater than about 20 bases, yet more preferably greater than about 50 bases and optimally greater than about 100 bases in length. The sequence is preferably at least about 70% identical, more preferably at least about 80% identical, more preferably still at least about 90% identical, even more preferably at least about 95% identical and optimally at least about 98% identical to the RhCG or Rhcg nucleotide sequence.

In a second embodiment the invention provides an isolated nucleic acid which hybridizes under stringent conditions with a nucleic acid having the nucleotide sequence of SEQ ID No.: 1, or SEQ ID No.: 2, or a sequence complementary to SEQ ID No.: 1, or SEQ ID No.: 2. This nucleotide sequence is preferably greater than about 30 bases in length, more preferably greater than about 50 bases, more preferably still greater than about 75 bases, yet more preferably greater than about 100 bases and optimally greater than about 150 bases long. The nucleotide sequence may be 1000 bases or more in length and may even extend up to several kilobases in length as shown in FIG. 1.

In another embodiment, the invention provides a nucleic acid molecule comprise a nucleotide sequence homologous to the RhCG sequence (SEQ ID No.: 1) or the Rhcg sequence (SEQ ID No.: 2) encoding a protein comprising an amino acid sequence at least 60% identical to at least a portion of the RhCG glycoprotein (SEQ ID No.:3) or to the Rhcg glycoprotein (SEQ ID No.: 4). The stretch of amino acids with 60% identity is preferably at least about 8-10 amino acids in length, more preferably at least about 12-15 amino acids, more preferably still at least about 18-20 amino acids, and optimally at least about 25-30 amino acids. The stretch of sequence with 60% overall identity may, however, align with a substantial portion, or even the entire RhCG or Rhcg length of 479 or 498 amino acids, respectively.

In yet another embodiment the isolated nucleic acid of the invention comprises a nucleotide sequence having at least about 60% sequence identity with the RhCG gene sequence (SEQ ID No.: 1) or the Rhcg gene sequence (SEQ ID No.: 2), which nucleotide sequence encodes a protein having RhCG or Rhcg activity. In one aspect, the activity is transporter activity. The transporter activity may be any transporter activity characteristic of a 12-transmembrane transporter protein such as an ion transporter activity. The ion transporter activity may be any ion transporter activity such as, for example, an NH₄⁺ ion transporter activity.

These nucleic acids may be subjected to any of the well known methods mutagenesis, including for example, site directed mutagenesis to alter the characteristics of the transporter activity. For example, the mutant RhCG or Rhcg gene may have altered transporter kinetics. Alternatively, the mutant may exhibit altered specificity for the ion or molecule transported.

Also provided are fragments of the nucleic acid molecule homologous to the RhCG sequence (SEQ ID No.: 1) or the Rhcg sequence (SEQ ID No.: 2) encoding a protein having RhCG or Rhcg activity. In one aspect, the activity is transporter activity. The transporter activity may be any transporter activity characteristic of a 12-transmembrane transporter protein such as an ion transporter activity. The ion transporter activity may be any ion transporter activity such as an NH₄⁺ ion transporter activity.

Further provided are fragments of the nucleic acid molecule homologous to the RhCG sequence (SEQ ID No.: 1) or the Rhcg sequence (SEQ ID No.: 2) encoding a protein having an epitope of RhCG or an epitope of Rhcg. The epitope is preferably comprised of an amino acid sequence of at least about 4-6 amino acids in length, though it may comprise 8 or more amino acids, or may be a conformational epitope of sequence of non-contiguous amino acids. In one aspect, the epitope is an epitope of RhCG. The epitope of RhCG may be any epitope of RhCG, including for example an epitope of the C-tail such as an epitope within the sequence from amino acid 417 to 479.

These nucleic acids and fragments are useful as probes for the Rh type C glycoprotein genes and homologues in many ways, including for example in Northern blots to identify tissues such as kidney and testis in which mRNAs from the RhCG and Rhcg genes are highly expressed. Further these probes are useful in detection of Rh type C glycoprotein genes in recombinant vectors and host cells transformed with such recombinant vectors.

The invention also provides isolated proteins and peptides comprising an amino acid sequence at least 60% identical to the RhCG glycoprotein (SEQ ID No.: 3) or to the Rhcg glycoprotein (SEQ ID No.: 4). The isolated protein or peptide may comprise an amino acid sequence encoded by a nucleic acid molecule of the invention described above. Further the isolated protein or peptide may comprise an epitope that is specifically bound by an antibody which specifically binds the RhCG glycoprotein or by an antibody which specifically binds the Rhcg glycoprotein. The isolated protein or peptide of the invention may be glycosylated or unglycosylated.

In yet another embodiment the invention provides a fusion protein comprising a fragment of the protein or peptide homologous to the RhCG glycoprotein (SEQ ID No.: 3) or to the Rhcg glycoprotein (SEQ ID No.: 4). The fusion protein may comprise an epitope of RhCG glycoprotein or Rhcg glycoprotein. Alternatively, the fusion protein may have transporter activity as described above. In another alternative, the fusion protein may comprise a detectable peptide or a capture epitope. Examples of detectable peptides include GFP and enzymes such as luciferase, β-lactamase, phosphatase or peroxidases. Suitable capture epitopes include myc-tags, polyhistidine tags and GST.

The invention further provides recombinant vectors comprising the RhCG and Rhcg nucleic acid molecules and nucleic acid homologues of RhCG and Rhcg described above.

Yet further, the invention provides host cells transformed with the recombinant RhCG and Rhcg vectors described above. The host cell may be a mammalian host cell, an insect host cell, a fungal host cell, a yeast host cell or a bacterial host cell.

Still further, the invention provides antibodies that specifically bind to an epitope of RhCG glycoprotein or to an epitope of Rhcg glycoprotein. The antibody may be a monoclonal antibody or a polyclonal antibody. The antibody may bind any epitope of RhCG or Rhcg such as an epitope of the N-terminal extracellular region, an extracellular loop, an intracellular loop or the C-terminal intracellular region.

In a preferred embodiment, the anti-RhCG or anti-Rhcg antibody is an antibody that binds an epitope of an Rh type C glycoprotein, or fragments of such an antibody, such as F(ab)₂ fragments, Fv fragments, single chain antibodies and other forms of “antibodies” that retain the ability to bind to an Rh type C glycoprotein. The antibody may be chimerized or humanized. In this specification, a chimerized antibody comprises the constant region of a human antibody and the variable region of a non-human antibody, such as a murine antibody. A humanized antibody comprises the constant region and framework variable region (i.e. variable region other than the hypervariable region) of a human antibody and the hypervariable region of a non-human antibody, such as a murine antibody. Of course, the antibody can be any other type of antibody derivative, such as a human antibody selected or screened from a phage display system or produced from a xenomouse.

These antibodies or antibody fragments that retain binding ability may be used in Western blots, ELISAs or in immunohistochemical assays to identify the non-erythroid tissues, particularly kidney and testis, that express the RHCG or Rhcg glycoproteins.

Also provided are Rh gene specific nucleotide probes including those having the nucleotide sequences of SEQ ID No.: 9, SEQ ID No.: 10, SEQ ID No.: 11, SEQ ID No.: 12, SEQ ID No.: 13, SEQ ID No.: 14, SEQ ID No.: 15, SEQ ID No.: 16, SEQ ID No.: 17, SEQ ID No.: 19, SEQ ID No.: 20, SEQ ID No.: 21, SEQ ID No.: 22, SEQ ID No.: 23, SEQ ID No.: 24 and SEQ ID No.: 25.

Isolated nucleic acid molecules comprising a functional RhCG regulatory region or a functional Rhcg regulatory region are also provided. The isolated nucleic acid may have a functional RhCG regulatory region comprising a fragment of the nucleotide sequence of SEQ ID No.: 7. Alternatively, the isolated nucleic acid having a functional Rhcg regulatory region may comprise a fragment of the nucleotide sequence of SEQ ID No.: 8.

Also provided are hybrid genes under Rh type C gene regulation comprising an upstream nucleic acid regulatory sequence of the RhCG gene and a coding sequence of a gene to be regulated. The upstream nucleic acid regulatory sequence may comprise a nucleic acid sequence from upstream of RhCG (SEQ ID No.: 7). Alternatively, the hybrid gene according may comprise a nucleic acid regulatory sequence from upstream of Rhcg (SEQ ID No.: 8).

The invention also provides a method of detecting an Rhcg or an RhCG glycoprotein in a sample, said method comprising: providing a sample to be tested, contacting the sample with an antibody that specifically binds to an epitope of RhCG glycoprotein or an Rhcg glycoprotein under conditions suitable for binding, assessing the specific binding to the antibody, and thereby detecting the presence of an epitope of Rhcg or RhCG glycoprotein in the sample. Standard detection methods useful in these assays, such as radioimmunoassays (RIAs), enzyme linked immunosorbent assays (ELISA), fluorescence resonance energy transfer (FRET) and scintillation proximity assays (SPA) are well known to the artisan. This method may be adapted to allow measurement or monitoring of the level of RhCG or Rhcg in a sample. In such methods the amount of bound RhCG or Rhcg detected is assessed, measured, or otherwise determined and may be compared with the amount bound in another sample or a reference standard. Similarly, samples taken at a different times may be compared.

The invention further provides a method of detecting an Rhcg or RHCG nucleotide sequence in a sample, said method comprising: providing a nucleic acid sample, contacting the sample with a nucleic acid probe that hybridizes to a nucleotide sequence having at least 75% sequence identity with SEQ ID No.: 1 or SEQ ID No.: 2, under conditions suitable for hybridization, detecting the nucleic acid probe hybridized to the sample, and thereby detecting the presence of an Rhcg or RhCG nucleotide sequence in the sample. The assay may be performed with an immobilized sample or probe, and include a washing step to remove unhybridized probe or sample, or the assay may be performed in an homogeneous solution phase.

### GENERAL METHODS

Labelling of nucleic acid probes—Methods for labelling oligonucleotide probes have been described. See for example, Leary et al., Proc. Natl. Acad. Sci. USA 80:4045 (1983); Renz and Kurz, Nucl. Acids Res. 12:3435 (1984); Richardson and Gumport, Nucl. Acids Res. 11:6167 (1983); Smith et al., Nucl. Acids Res. 13:2399 (1985); Meinkoth and Wahl, Anal. Biochem. 138:267 (1984); and Ausubel, F. M. et al. (Eds.) Current Protocols in Molecular Biology, John Wiley & Sons, Inc., New York, 1999.

The detectable moiety employed as a label for the nucleic acid probe may be radioactive. Some examples of useful radioactive labels include ³²P, ¹²⁵I, ¹³¹I, ³⁵S, ¹⁴C, and ³H. Use of radioactive labels have been described in U.K. patent 2,034,323, U.S. Pat. Nos. 4,358,535, and 4,302,204. Some examples of non-radioactive moieties useful as labels include enzymes, chromophores, heavy atoms and molecules detectable by electron microscopy, and metals detectable by their magnetic properties.

Some useful enzymatic labels include enzymes that cause a detectable change in a substrate. Some useful enzymes and their substrates include, for example, horseradish peroxidase (pyrogallol and o-phenylenediamine), beta-galactosidase (fluorescein beta-D-galactopyranoside), and alkaline phosphatase (5 -bromo- 4-chloro3-indolyl phosphate/nitro blue tetrazolium). The use of enzymatic labels have been described in U.K. 2,019,404, EP 63,879, in Ausubel, F. M. et al. (Eds.), Current Protocols in Molecular Biology, John Wiley & Sons, Inc., New York (1999), and by Rotman, Proc. Natl. Acad. Sci. USA 47:1981-1991(1961).

Site directed mutagenesis—Synthetic or naturally occuring DNA may be subjected to mutagenesis at predetermined locations and screened for clones expressing the mutated protein; See, for example, Zoller and Smith, Nucl. Acids Res. 10:6487-6500 (1982); Methods Enzymol. 100:468-500 (1983); DNA 3:479-488 (1984); Kunkel, T. A. et al., Methods Enzymol. 154:367-382, Academic Press, Inc., New York (1987); Uhhnann, E., Gene 71:29-40 (1988); Myers, R. M. et al., Science 229:242-246 (1985); Myers, R. M. et al., Methods Enzymol. 155, 501-527, Academic Press, Inc., New York (1987); and Current Protocols in Molecular Biology, Ausubel, F. M. et al. (Eds.), John Wiley & Sons, Inc., New York, (1999).

Sequence comparisons: Alignments of nucleic acid coding sequences and of amino acid sequences of proteins, and assessment of the percent homologies (including identities and similarities) may also be performed to provide further examples of sequences of the present invention. For searches and alignments of sequences the BLAST homology search program is available on the internet at http://www.ncbi.nlm.nih.gov giving % homology determinations according to the above defined criteria.

### EXAMPLES

### MATERIALS AND EXEMPLIFIED METHODS

Cloning of Mouse Rhcg and Human RHCG cDNAs—Using mouse Rhag cDNA (12) for a BLAST search (16), a mouse testis expressed sequence tag (AA063867) of high homology was detected. Sense (s) or antisense (a) gene-specific primers (GSPs) were designed to obtain full-length Rhcg cDNA. For 5′-RACE (17), 1 mg of mouse testis total RNA was primed with GSP-E7a (GAAGGAATGGACTATCCCTGGCTC) This primer is SEQ ID No.: 9.

The cDNA was tailed with dCTP using the supplier's protocol (Life Technologies, Inc.) and amplified by two-round PCR with supplied adapter primers and GSPs (E6 a1, GGTGGAGAAAATGCCGCAGAA, SEQ ID No.: 10; E6a2, AACCCCACGATGAGAGCGCCGTAA, SEQ ID No.: 11). A 1.1-kb cDNA was purified and sequenced; 3′-RACE (17) was similarly carried out with GSPs (E7s1, CCATTCCTGGAGTCCCGCCTT; SEQ ID No.: 12 and E7s2, CGCATCCAGGACACATGTGGCA, SEQ ID No.: 13).

To clone RHCG, degenerate primers (RhCG-d1, TC(C/T)ATGAC(C/T)ATCCA(C/T)ACATT(C/T)GG, SEQ ID No.: 14; RhCG-d2, CAG(A/G)TT(A/G)TG(A/G)ATGCC(A/G)CATGTGTC, SEQ ID No.: 15) corresponding to conserved Rhcg regions (codons 183-190 and 337-344) were used in reverse transcription-PCR of human kidney total RNA. A 480-bp cDNA was purified and sequenced. After comparison with Rhcg open reading frame, GSPs were designed and used to derive full-length RHCG cDNA, as described above. Other molecular biological procedures followed standard methods (18).

Comparison of RHCG, Rhcg, and Other Homologues—The amino acid sequences of RhCG, Rhcg and other Rh homologues were aligned by means of Clustal W (19) (MegAlign software, DNA*). The hydropathy plots were obtained using the Kyte-Doolittle method (20).

RHCG Expression Constructs—The full-length RHCG cDNA was cloned in pCR2.1 vector (Invitrogen) using the following GSPs: E10a(XhoI), CCGCTCGAGCTAGGGTACCAAGGGTACCGA, SEQ ID No.: 16; E1s(Bg-III), GAAGATCTAGCATGGCCTGGAACACCA, SEQ ID No.: 17. All constructs were derived from this master template with Pfu DNA polymerase and were verified to be free of mutations by sequencing. To tag the green fluorescence protein (GFP), RhCG was amplified by GSP-E1s(BgIII) plus E10a(XhoI) or E1s(BgIII) plus E10a(SaII) and subcloned in the pEGFP-C1 or pEGFP-N3 vector (CLONTECH). For translation and expression studies, RHCG was cloned in pYES2 or pcDNA3.1/MycHisA (Invitrogen) by BamHI and XhoI double digestion. To express the RhCG C-tail (amino acids 417-479, SEQ ID No.: 18), its coding region was amplified by GSP-E9s(BamHI) (CGGGATCCAGATTACCATTCTGGGGAC, SEQ ID No.: 19) plus E10a(XhoI) (SEQ ID No.: 16) and cloned separately into vectors pGEX-4T-1 (Amersham Pharnacia Biotech) and pET30a(+) (Novagen).

Production of Polyclonal Antisera for RhCG C-tail—The RhCG C-tail was expressed in E. coli BL21 (0.3 mM isopropyl-1-thio-β-D-galactopyranoside at 30° C. for 4 h) as a glutathione S-transferase fusion protein or a His₆-tagged peptide. After sonication, glutathione S-transferase-RhCG was purified on glutathione Sepharose 4B, and ˜300 mg was emulsified with adjuvant and injected into rabbits five times (21). Antisera were affinity-purified by passing through a glutathione S-transferase column (Pierce) and then a Ni-NTA column (Qiagen) bound with His-tagged RhCG tail. After washing, the antibodies were eluted with 4 M MgCl2 and dialyzed in 1X phosphate-buffered saline at 4° C. The IgG fractions showed binding to recombinant RhCG C-tails only.

Screening of Genomic Clones and Definition of Exon/Intron Boundaries—Bacterial artificial chromosome (BAC) clones of RHCG or Rhcg were screened by PCR. The GSPs for RHCG (E6s, CGTGGGTACCGCTGCTGAGAT, SEQ ID No.: 20; E7a, TATGATGCCAGGAATGCCA TGCAG, SEQ ID No.: 21) gave a 360-bp band. The GSPs for Rhcg (E6s, GCTCTCATCGTGGGGTTCTTCTGC, SEQ ID No.: 22; E7a, CAGGTTGTGRATGCCACATGTGTC, SEQ ID No.: 23) gave a 310-bp band. BAC DNA was fingerprinted by exon PCR and mapped by Southern blotting. Exon/intron boundaries were amplified in two steps from genomic libraries (5, 12) and sequenced. Exon/intron junctions were defined by alignment with cDNA sequences. Introns were amplified from BAC DNA, and their sizes were estimated by gel electrophoresis.

5′-RACE and 5′ Genomic Walking—5′-RACE was performed as mentioned above. The 5′ region of RHCG or Rhcg gene was amplified from the human or mouse genomic libraries (5, 12) using adaptor primers and exon 1 GSPs. The PCR products were purified and sequenced.

Chromosomal Mapping of RHCG and Rhcg Genes—Fluorescence in situ hybridization to interphase chromosomes (22) was used to map human RHCG. The DNA from BAC clone 300M8 or 2160I19 was labeled as probes. Rhcg was assigned using Jackson BSS interspecific backcross ((C57BL/6JEiXSPRET/Ei)FIXSPRET/Ei) (23). The HhaI restriction site was present in Rhcg intron 4 of C57BL/6JEi but not in intron 4 of SPRET/Ei. Rhcg linkage was defined by HhaI digestion of intron 4 fragments in all 94 progenies amplified with GSPs (CTCACAGTGACCTGGATCCTCTAC, SEQ ID No.: 24 and CATATCCAACTTGCCCTTCTTGTG, SEQ ID No.: 25).

Northern Blot Analysis and RNA in Situ Hybridization—Two sets of Northern blots (CLONTECH) were hybridized to RHCG or Rhcg cDNA. The RHCG (codon 250-437) and Rhcg (codon 194-377) probes were 563 and 552 bp in size, respectively. As a control, the β-actin probe was used.

RNA in situ hybridization to mouse embryos and tissues was carried out using standard protocols, as described (24). A 384-bp sequence covering the 3′ region of Rhcg cDNA (nucleotides 1110-1494) was subcloned in pCRScript Sk(1) vector (Stratagene) and used to prepare ³²P-labeled probes. The antisense and sense probes were generated by in vitro transcription of the BamHI- and NotI-linearized Rhcg plasmids by T7 and T3 RNA polymerases, respectively.

Transfection of RHCG cDNA and Confocal Imaging of RhCG Protein—HEK293 and HeLa cells (ATCC) were grown at 37° C. in Dulbecco's modified Eagle's medium containing 10% fetal calf serum (Life Technologies, Inc.) under 5% CO₂. After 24 h, cells were transferred to either a six-well plate or cover glass and transfected by LipofectAMINE (Life Technologies, Inc.). For transient expression, 1 mg of RHCG cDNA was used to transfect 3×10⁵ HEK293 or HeLa cells. For stable selection, RHCG.Myc/HisA plasmid (1 mg, Pvul cut) was used to transfect 3×10⁵ HEK293 cells/well in a six-well plate. Stable cell lines were selected in Dulbecco's modified Eagle's medium (G418, 800 mg/ml) and clonally isolated by standard procedures (18). For confocal imaging, 1 mg of RhCG-GFP or GFP-RhCG plasmid was transfected in 3×10⁵ HEK293 or HeLa cells plated onto a 35-mm coverglass (MatTek) and cultured for 24 h. GFP was excited at 488 nm with an argon laser, and the light emitted between 506 and 538 nm was recorded for fluorescein isothiocyanate. Images were collected using a Bio-Rad MRC 600 confocal scan head on a Nikon Eclipse 200 microscope with a 60XN.A.1.4 planapo infinity corrected objective. The captured three-dimensional images were processed with Adobe Photoshop (version 4.0).

In Vitro Translation and N-Glycosylation—To define the biochemical features of RhCG, in vitro translation and posttranslational processing were carried out. As templates, RhCG plasmids of pYES or pcDNA3.1/MycHisA were used in a transcription-coupled translation system (Promega) with [³⁵S]methionine (15 mCi/ml, Amersham Pharmacia Biotech). Labeled RHCG was analyzed by 12% SDS-PAGE (25) after incubation with or without canine pancreatic microsomal membranes (CPMM) (Promega).

Western Blot and N-Glycanase Analysis of RhCG—Cellular fractionation and isolation of membrane vesicles from stable HEK293 cells were done as described with minor modification (26). Protein was resuspended at 1 mg/ml in ice-cold buffer (10 mM HEPES, pH 7.5, 1 mM MgCl2 , 250 mM sucrose). RBC ghosts were prepared as described (27). 8 mg of HEK293 membranes and 50 mg of RBC membranes, treated with or without PNGase F (New England BioLabs), were analyzed by 12% SDS-PAGE (25). Western blots were probed by purified anti-RhCG (1:1, 500) or HPR-conjugated anti-Myc monoclonal antibody (1:5,000) (Invitrogen). The anti-RhCG probed blot was stained with horseradish peroxidase-linked donkey anti-rabbit IgG (1:5,000) (Amersham Pharnacia Biotech) and visualized by a chemiluminescent kit (Pierce). The anti-Myc probed blot was visualized directly with the above detection system.

Characterization of human RhCG and mouse Rhcg glycoproteins and genes.

Nucleotide Sequences of RHCG and Rhcg cDNAs—Sequencing revealed that RHCG and Rhcg cDNAs, 1952 and 2097 bp long, respectively, share an overall 68.8% identity at the nucleotide level, forming an orthologous pair. The variation in the sequence is mainly in the 5′-UTR and 3′-end of open reading frames (FIG. 1). RHCG and Rhcg have a consensus polyadenylation signal but lack the typical Kozak sequence (28) and stop codon preceding the first in-frame AUG codon, two features common to the erythroid Rh genes (5, 12). However, RHCG and Rhcg are G/C-rich (RHCG, 57.5%; Rhcg, 55.4%; versus RHAG, 42.6%; Rhag, 41.8%) and have completely different 5′- and 3′-UTR sequences.

The Primary Structure and Predicted Topology of RhCG and Rhcg Proteins—The open reading frame of human RHCG encodes a 53-kDa, 479-amino acid polypeptide, whereas that of mouse Rhcg encodes a 55-kDa, 498-amino acid polypeptide (FIG. 1). The RhCG and Rhcg proteins are highly conserved, sharing 77.2% identity and 90.4% similarity with regard to their primary structures. RhCG differs from Rhcg by three small deletions: one serine at position 51 and two amino acid stretches in the extreme C-terminal region are absent in RhCG (FIG. 1). The majority of variations are conserved amino acid changes, with ˜50% of them being clustered at the C-terminal positions of 360/361.

The RhCG (pI=6.2) and Rhcg (pI=6.5) proteins are negatively charged at physiologic pH and are composed mainly of hydrophobic (44%) and polar (25%) amino acids. Hydropathy analysis (20) showed that they share identical topology and charge distribution, each spanning the lipid bilayer 12 times, with both N and C termini facing the cytoplasm (FIG. 2). The 12-TM fold and its cognate signatures, including the charged residues (Asp 129 and Glu 166), are conserved and reminiscent of other homologs, i.e. the RhAG/Rhag (4, 12) and RhBG/Rhbg pairs (FIG. 3). However, RhCG and Rhcg are distinct in that they have a much elongated C-terminal segment and unique N-terminal and exoloop sequences. There are also three N-glycosylation NX(S/T) motifs in RhCG and two in Rhcg (FIG. 1), suggesting that these proteins are expressed as polytopic gly-coproteins. Nevertheless, except for 48 NLS 50 (RhCG) (FIG. 2) or 48 NIS 50 (Rhcg), other NX(S/T) motifs were predicted to reside in the TM or in the cytoplasmic domain and thus are unlikely to become glycosylated in vivo.

Relationship of RhCG/Rhcg with Other Rh Protein Homologs—Along with the identification of RhCG and Rhcg, we also isolated cDNAs encoding new Rh homologues from model organisms and nonerythroid tissues. Multiple sequence alignment yielded a dendrogram in which the known Rh homologues are clustered in three distinct groups: primitive, erythroid, and nonerythroid (FIG. 3). RhCG and Rhcg were placed as a pair in the nonerythroid subgroup that also includes Homo sapiens RhBG, Mus musculus Rhbg, Danio rario Rhg, and Geodia cydonium Rhg. All these proteins are polytopic glycoproteins that are likely to contain a single N-glycan on their exoloop 1.

As shown by pairwise amino acid sequence comparison, RhCG and Rhcg are most closely related to the nonerythroid RhBG/Rhbg pair (51.7-52.3% identity) and erythroid RhAG/Rhag pair (45.4- 50.9% identity) (FIG. 3). This degree of overall identity is largely attributable to conserved TM segments (particularly TM2-11) and their immediately adjacent amino acids, suggesting that these domains define an important function. Significantly, RhCG and Rhcg are much less similar to antigen carriers, RhCE/D, or Rhced (24.4-28.5% identity). Considering their relationship with the primitive subgroup, RhCG and Rhcg are more closely related to the fruit fly homologue Dm.Rhp, with which they share a 37% overall sequence identity (FIG. 3). These results suggest that the RHCG/Rhcg pair originated from early primitive gene precursors and was subjected to an independent evolutionary pathway following its duplication and separation from erythroid members.

Chromosomal Location of RHCG and Rhcg Genes—The chromosomal location of the human RHCG gene was mapped by fluorescence in situ hybridization using the two BAC genomic clones as probes. The result showed that RHCG is located at 15q25 of chromosome 15 (FIG. 4A). Using RHCG as a query for BLAST search, we detected an exactly matched expressed sequence tag (U75833) from the human CEPH YAC clone that had been mapped at 15q26 (29). These data establish a separate location of RHCG from the erythroid RHCED and RHAG loci that are mapped at chromosomes 1p34-36 and 6p11-21.1, respectively (3, 4).

The mouse Rhcg gene was mapped by linkage analysis (FIG. 4B). Rhcg is nonrecombinant to the D7Xrf229 locus, with a LOD score of 28.3. This linkage placed Rhcg distal to Gnb2-rs1 but proximal to Pcsk3 on the long arm of chromosome 7, a region showing conserved synteny with human 15q25 containing RHCG gene (FIG. 4A). Notably, this locus map is also separate from the erythroid Rhced and Rhag loci that are localized to chromosomes 4 and 17, respectively (12).

Genomic Organization and Exon/Intron Structures—BAC clones retaining human RHCG or mouse Rhcg gene were isolated and characterized in order to delineate their structural organization. As shown in FIG. 5, the two genes share a nearly identical organization, each having 11 exons that range from 74 to 187 bp and 10 introns that range from 0.2 to 8.8 kb. In both RHCG and Rhcg genes, exon 11 occurs as a noncoding 3′-UTR segment, a unique feature that distinguishes from all other Rh homologs. Their internal exons 2-9 are identical in size, and their exons 2-6 are arranged in the same fashion as exons 2-6 of RHAG and Rhag genes (FIG. 5). Because exons 2-6 encode TM2-9 domains in both erythroid and nonerythroid homologs, their conservation in sequence and size implies strict control of the length of the corresponding TM domains and flanking loops. FIG. 6 shows the sequences of RHCG and Rhcg genes containing splice sites and exon/intron junctions. All 5′ donor and 3′ acceptor sites conform to the “GT-AG” rule and show consensus to the primate and rodent gene splicing signals (30). Of the 10 coding exons assigned, all but exons 6 and 10 are asymmetrical containing one (exons 1, 3, 4, 5, 7, and 9) or two (exons 2 and 8) split intercodons at the 5′, and/or 3′ exon/intron junction. The amino acids encoded by exon/exon boundaries are the same between RhCG and Rhcg, except for four conserved changes (FIG. 6). Such a division of coding exons is observed in mammals (10-12) but not in more primitive organisms.

Proximal Promoter Sequence and Transcription Start Site—Genomic walking revealed that the 5′ region upstream of the first ATG codon of RHCG or Rhcg contains multiple cis-acting elements known to bind various transcription factors (FIG. 7). This proximal sequence is highly enriched in CpG dinucleotides (3′ CpGs out of 443 bp for RHCG and 25 CpGs out of 279 bp for Rhcg), a hallmark of CpG islands that often overlap with generic promoters (31). The sequence is also highly asymmetrical in strand composition alternating with pyrimidine and purine stretches. Notably, these features and many cis-acting motifs are absent from erythroid RH promoters (5, 6, 12, 32-34).

Sequencing of the 5′-RACE products identified −24A and −126A as the major transcription start sites in the RHCG and Rhcg genes, respectively (FIG. 7). The sites were determined by using total RNA isolated from the human kidney and mouse testis tissues, and this mapping result was consistent with the beginning of 5′-UTR sequence observed in the full-length cDNAs (FIG. 1). The fact that no in-frame ATG occurs in the genomic sequence further upstream of the transcription start site (FIG. 7) also supports the predicted AUG codon as the translation initiation signal (FIG. 1).

Tissue Expression of RHCG and Rhcg—The expression of RHCG and Rhcg was assessed by Northern blot analysis (FIG. 8) and by in situ hybridization (FIG. 9). In human adult tissues, a 2.0-kb RHCG transcript was found abundantly expressed in the kidneys and also in the brain, testis, placenta, pancreas, and prostate (FIG. 8A). (Its apparent lower expression in the testis was probably caused by an uneven sampling of testicular tissues for poly(A)+ RNA preparation.) In human fetal tissues, the kidney was the only organ found to express RHCG transcripts.

In mouse adult tissues, Rhcg was shown to be also highly expressed in kidney and testis but not in other tissues, e.g. brain (FIG. 8B). Furthermore, no Rhcg transcript was detected in embryos of 7-19 days of gestation nor in erythroid tissues. Taken together, the results indicate that RHCG or Rhcg is largely expressed at very late stages of development and with limited tissue specificities. The results from RNA in situ hybridization further helped refining of the sites of Rhcg expression in mouse tissues (FIG. 9). In mouse testis, Rhcg was abundantly expressed in the seminiferous tubules (FIG. 9B), the main components of the adult testicle that produce spermatozoa (35). Higher magnification revealed that the Rhcg signals decorated the complex stratified epithelium (FIG. 9C), a lining of the seminiferous tubules known to contain spermatogenic cells and supporting Sertoli cells (35). In mouse kidney, Rhcg was broadly and abundantly expressed in both the renal cortex and the medula (FIG. 9D). The signals exhibited a radially distributed pattern along their length that corresponded to the parallel alignment of the myriad minute uriniferous tubules that form the functional renal units (35). At higher magnification, the Rhcg signal was primarily confined to the epithelial linings of the collecting tubules (FIGS. 9, E and F). These results point toward specific expression of Rhcg in the tubular structures of the testis and the kidney.

Cellular Localization of RhCG by Confocal Imaging Analysis—Confocal microscopy (FIG. 10) revealed that the fluorescence signal was confined to the plasma membrane of cells transfected with RhCG-GFP constructs (B, C, E, and F), whereas it was evenly distributed in the cytoplasm of cells transfected with GFP vector alone (A and D). The membrane localization was not dependent on the orientation of GFP in the fusion constructs or on the type of cells used in the transfection assay. These results imply that the RhCG protein is normally destined to the plasma membrane.

In Vitro Translation and N-Glycosylation of RhCG Protein—To assess ER translocation and early processing events during biosynthesis, RhCG was translated in vitro, in either the absence or presence of CPMM, and analyzed by SDS-PAGE. In the absence of CPMM, in vitro translated RhCG, with or without a c-Myc tag, migrated as a single band with a molecular mass less than the predicted molecular mass of 53 kDa (FIG. 11A). This mobility anomaly is also seen in erythroid homologues and is most likely the result of the highly hydrophobic nature of these proteins (33). In the presence of CPMM, the RhCG proteins exhibited a slower migration pattern suggesting that they may be differentially glycosylated (FIG. 11 B). The observed size differences between the CPMM-treated and untreated protein products can be accounted by the size of a single complex N-glycan. These in vitro studies indicated that RhCG could be glycosylated.

Western Blot and N-Glycanase Analysis of RhCG Protein—To establish the in vivo expression of RhCG as a glyco-protein, membrane proteins were isolated from HEK293 cells stably transfected with the RHCG.Myc/His construct. Western blots probed with the RhCG C-tail polyclonal antiserum showed a major protein band with a molecular mass of ˜58 kDa (FIG. 12A, lanes 3 and 4); this size was slightly larger than that of in vitro glycosylated RhCG (FIG. 11 B, lane 4). After N-glycanase treatment, the size of membrane-bound RhCG was decreased to 46 kDa (FIG. 12A, lanes 5 and 6), which was equivalent to that of in vitro translated but unglycosylated RhCG (FIG. 11A, lane 6). This result indicated that the same AUG initiation codon could be used for RhCG synthesis in both in vivo and in vitro. Because the stably expressed RhCG protein was expected to carry a Myc/His tag at the C-terminal end, anti-Myc monoclonal antibody was used to probe the same Western blots. This analysis revealed the same banding pattern (FIG. 12B), confirming that RhCG exists as a membrane glycoprotein of which the N-glycan is most probably attached to the 48 NLS 50 sequon on exoloop 1 (FIG. 2).

Clustal analyses of known Rh homologues define RhCG and Rhcg as the first new members of the nonerythroid Rh subfamily in vertebrate species, including all mammals. The detailed studies concerning the genetic and biochemical aspects of RhCG/Rhcg revealed common features as well as distinct differences between the erythroid and non-erythroid Rh homologues. Collectively these results provide novel insights into the molecular evolution, structural conservation, tissue-specific expression, and biological function of the Rh family of genes and proteins.

The many homologous sequences assembled suggests the existence of a Rh superfamily, which to date consists of five and four discrete gene members in humans and mice, respectively. Based on their genomic synteny, sequence identity, and tissue specificity, the human and mouse homologues can be divided into four orthologous groups, i.e. two erythroid gene pairs (RHAG/Rhag and RHCED/Rhced) and two nonerythroid gene pairs (RHCG/Rhcg and RHBG/Rhbg). Notably, all the RH loci harbor single copy genes, except for human RHCED (37), and all have a different map location, although each orthologous group shows comparable chromosomal synteny. By contrast, there is only one Rh gene in the unicellular organism slime mold (2), two tightly linked copies in C. elegans (15), and three discrete members in the zebrafish. These observations suggest that both the erythroid and nonerythroid branches came into existence before mammalian radiation and that their multiplicity occurred mainly via intergenic translocation events. This pattern of RH duplication is likely to have produced functional novelty in a different temporal and spatial context within the organism, given changes in both coding sequence and regulatory elements of the gene (5-7, 10-12, 32-34).

Compared with erythroid homologues, both primitive and nonerythroid members, including RhCG and Rhcg, are structurally much closer to RhAG/Rhag than they are to RhCED/Rhced. By excluding the more distant RhCED/Rhced group, a membrane fold has been identified, which is characterized by shared signatures conserved among human/mouse RhCG/Rhcg, RhBG/Rhbg, and RhAG/Rhag pairs. This relationship conforms to the approximate equidistance between the erythroid and nonerythroid branches and extends the observation that the RHCED series originated from an Rhag ancestor gene but evolved at a much increased rate (11, 38). The slower divergence of RHAG and the faster evolution of RHCED imply an adapted functional specification that pertains to Rh complex assembly in the RBC membrane through heteromeric interactions. Without RhAG expression, as observed in the regulator type of Rhₙᵤₗₗ disease (40, 41), the RhCE/D proteins and their associated antigens are not dispositioned at the cell surface. In sharp contrast, such heteromeric interaction is not a prerequisite for surface expression of RhCG, as RHCG could by itself reach the plasma membrane, whether transfected into homologous or heterologous cells.

Besides their capability of being expressed in heterologous cells, RHCG and Rhcg differ from erythroid members in the following features:

1) They each have a larger exoloop 6 and much longer cytoplasmic C-terminal sequence that is rich in Pro and Ser residues.

2) RHCG and Rhcg are each unique, having a CpG island-rich promoter and an extra 3′-UTR exon.

3) The human RHCG gene is mapped at 15q25 and close to the type I tyrosinemia disease locus (42), although its phenotypic relationship with the latter is unknown.

4) The onset of Rhcg expression occurs very late in development, contrary to the early coexpression of Rhag and Rhced that parallels erythroid differentiation at embryonic stages (12).

5) Compared with the erythroid restriction of Rhag/Rhced (12), RHCG or Rhcg is abundantly and broadly expressed in the kidney and in the testis.

6) Although residing in the plasma membrane, as erythroid Rh proteins, Rhcg is concentrated in the epithelial linings of the tubular structures, the kidney collecting tubules, and the testis seminiferous tubules.

Rh proteins have long been thought to possess a transport activity due to their characteristic TM fold and associated morphological and physiological changes in Rhₙᵤₗₗ disease (2, 7). Prior studies suggested that Rh might function as an ATP-dependent phosphatidylserine transporter (phosphatidylserine flipase) of the RBC membrane (43, 44). Nonetheless, two lines of evidence disfavor this proposal: 1) Rh proteins lack the ATP-binding cassette (ABC) (2, 7); and 2) Rhₙᵤₗₗ cells have a normal phosphatidylserine transport activity (45).

The nucleotide sequences reported herein have been submitted to the GenBank™/EBI Data Bank and have the following accession numbers: AF193807, AF193808, AF193809, AF193810, AF183390, AF183391, AF183811, AF183812, AF209468, AF219981, AF219986, and AF238372, AF238377.

### REFERENCES

1. Mollison, P. L., Engelfriet, C. P., and Contreras, M. (1997) Blood Transfusion in Clinical Medicine, Blackwell Science, Oxford, England.

2. Huang, C.-H., Liu, Z., and Cheng, G. (2000) Semin. Hematol. 34, 150-165.

3. Cherif-Zahar, B., Mattei, M.-G., Le Van Kim, C., Bailly, P., Cartron, J.-P., and Colin, Y. (1991) Hum. Genet. 86, 398-400.

4. Ridgwell, K., Spurr, N. K., Laguda, B., MacGeoch, C., Avent, N. D., and Tanner, M. J. A. (1992) Biochem. J. 287, 223-228.

5. Huang, C.-H. (1998) J. Biol. Chem. 273, 2207-2213.

6. Matassi, G., Che' rif-Zahar, B., Raynal, V., Rouger, P., and Cartron, J.-P. (1998) Genomics 47, 286-293.

7. Agre, P., and Cartron, J.-P. (1991) Blood 78, 551-563.

8. Henderson, P. J. (1993) Curr. Opin. Cell Biol. 5, 708-721.

9. Marini, A. M., Urrestarazu, A., Beauwens, R., and Andre, B. (1997) Trends Biochem. Sci. 22, 460-461.

10. Blancher, A., and Socha, W. W. (1997) in Molecular Biology and Evolution of Blood Group and MHC Antigens in Primates (A. Blancher, J. Klein, W. W. Socha, eds) Springer Verlag, Heidelberg.

11. Huang, C.-H., Liu, Z., Apoil, P.-A., and Blancher, A. (2000, September) J. Mol. Evol., 51(3), 303-304.

12. Liu, Z., Huang, C.-H. (1999) Biochem. Genet. 37, 119-138.

13. Seack, J., Pancer, Z., Muller, I., and Muller, W. (1997) Immunogenetics 46, 493-498.

14. Wilson, R., Ainscough, R., Anderson, K., Baynes, C., Berks, M., Bonfield, J., Burton, J., Connell, M., Copsey, T., Cooper, J., et al. (1994) Nature 368, 32-38.

15. Huang, C.-H., Liu, Z., Cheng, G. J., and Chen, Y. (1998) Blood 92, 1776-1784.

16. Altschul, S. F., Madden, T. L., Schaffer, A. A., Zhang, J., Zhang, Z., Miller, W., and Lipman, D. J. (1997) Nucleic Acids Res. 25, 3389-3402.

17. Frohman, M. A., Dush, M. K. and Martin, G. (1988) P.N.A.S. U.S.A. 85, 8998-9002.

18. Sambrook, J., Fristch, E. F., and Maniatis, T. (1989) Molecular Cloning: A Laboratory Manual, 2nd Ed., Cold Spring Harbor Laboratory, Cold Spr. Harbor, N.Y.

19. Thompson, J., Higgins, D. and Gibson, T. (1994) Nucleic Acids Res. 22, 4673-4680.

20. Kyte, J., and Doolittle, R. F. (1982) J. Mol. Biol. 157, 105-132.

21. Harlow, E., and Lane, D. (1988) Antibodies: A Laboratory Manual 1st Ed., Cold Spring Harbor Laboratory, Cold Spring Harbor, N.Y.

22. Heng, H. H. Q., Squire, J., and Tsui, L.-C. (1992) P.N.A.S. U.S.A. 89, 9509-9513.

23. Rowe, L. B., Nadeau, J. H., Turner, R., Frankel, W. N., Letts, V. A., Eppig, J. T., Ko, M. S. H., Thurston, S. J., and Birkenmeier, E. H. (1994) Mamm. Genome 5, 253-274.

24. Hui, C.-c., and Joyner, A. L. (1993) Nat. Genet. 3, 241-246.

25. Laemmli, U. K. (1970) Nature 227, 680-685.

26. Fong, A. D., Handlogten, M. E., and Kilberg, M. S. (1990) Biochim. Biophys. Acta 1022, 325-332.

27. Huang, C.-H., Blumenfeld, O. O., Reid, M. E., Chen, Y., Daniels, G. L., and Smart, E. (1997) Blood 90, 391-397.

28. Kozak, M. (1987) Nucleic Acids Res. 15, 8125-8148.

29. Chumakov, I. M., Rigault, P., Le Gall, I., Bellanne-Chantelot, C., Billault, A., Guillou, S., Soularue, P., Guasconi, G., Poullier, E., Gros, I., et al. (1995) Nature 377 (suppl.), 175-297.

30. Shapiro, M. B., and Senapathy, P. (1987) Nucleic Acids Res. 15, 7155-7174.

31. Gardiner-Garden, M., and Frommer, M. (1987) J. Mol. Biol. 196, 261-282.

32. Cherif-Zahar, B., Le Van Kim, C., Rouillac, C., Raynal, V., Cartron, J.-P., and Colin, Y. (1994) Genomics 19, 68-74.

33. Huang, C.-H. (1996) Blood 88, 2326-2333.

34. Iwamoto, S., Omi, T., Yamasaki, M., Okuda, H., Kawano, M., and Kajii, E. (1998) Biochem. Biophys. Res. Commun. 243, 233-240.

35. Fawcett, D. W., and Raviola, E. (1994) A Text Book of Histology 12th Ed., Chapman & Hall, N.Y.

36. Moore, S., Woodrow, C. F., and McClelland, D. B. L. (1982) Nature 295, 529-531.

37. Carritt, B., Kemp, T. J., and Poulter, M. (1997) Hum. Mol. Genet. 6, 843-850.

38. Kitano, T., Sumiyama, K., Shiroishi, T., and Saitou, N. (1998) Biochem. Biophys. Res. Commun. 249, 78-85.

39. Ridgwell, K., Eyers, S. A., Mawby, W. J., Anstee, D. J., and Tanner, M. J. (1994) J. Biol. Chem. 269, 6410-6416.

40. Huang, C.-H., Cheng, G.-J., Liu, Z., Chen, Y., Reid, M. E., Halverson, G., and Okubo, Y. (1999) Am. J. Hematol. 62, 25-32.

41. Cherif-Zahar, B., Raynal, V., Gane, P., Mattei, M.-G., Bailly, P., Gibbs, B., Colin, Y., and Cartron, J.-P. (1996) Nat. Genet. 12, 168-173.

42. OMIM (1999) in The Human Genome Database Project. Johns Hopkins University, Baltimore, Md., http://www.gdb.org/omin/docs/omimtop.html.

43. Connor, J., and Schroit, A. J. (1988) Biochemistry 27, 848-851.

44. Schroit, A. J., Bloy, C., Conner, J., and Cartron, J.-P. (1990) Biochemistry 29, 10303-10306.

45. Smith, R. E., and Dalek, D. L. (1990) Blood 76, 1021-1027.

46. Marini, A. M., Soussi-Boudekou, S., Vissers, S., and Andre, B. (1997) Mol. Cell. Biol. 17, 4282-4293.

47. Kaiser, B. N., Finnegan, P. M., Tyernan, S. D., Whitehead, L. F., Bergersen, F. J., Day, D. A., and Udvardi, M. K. (1998) Science 281, 1202-1206.

48. Bult, C. J., White, O., Olsen, G. J., Zhou, L., Fleischmann, R. D., Sutton, G. G., Blake, J. A., Fitzgerald, L. M., Clayton, R. A., Gocayne, J. D. et al. (1996) Science 273, 1058-1073.

1. An isolated nucleic acid comprising a nucleotide sequence having at least 60% sequence identity with at least a portion of SEQ ID No.: 1 or SEQ ID No.: 2.

2. An isolated nucleic acid which hybridizes under stringent conditions with a nucleic acid having the nucleotide sequence of SEQ ID No.: 1, or SEQ ID No.: 2, or a sequence complementary to SEQ ID No.: 1, or SEQ ID No.: 2.

3. The isolated nucleic acid according to claim 1 encoding a protein comprising an amino acid sequence at least 60% identical to at least a portion of SEQ ID No.: 3 or at least a portion of SEQ ID No.: 4.

4. The isolated nucleic acid according to claim 1 encoding a protein having RhCG or Rhcg activity.

5. The isolated nucleic acid according to claim 4 wherein the RhCG or Rhcg activity is transporter activity.

6. The isolated nucleic acid according to claim 5 wherein the transporter activity is ion transporter activity.

7. The isolated nucleic acid according to claim 6 wherein the ion transporter activity is NH4+ ion transporter activity.

8. A fragment of the nucleic acid molecule according to claim 1 encoding a protein having RhCG or Rhcg activity.

9. The fragment of a nucleic acid molecule according to claim 8 wherein the RhCG or Rhcg activity is transporter activity.

10. The fragment of a nucleic acid molecule according to claim 9 wherein the transporter activity is ion transporter activity.

11. The fragment of a nucleic acid molecule according to claim 10 wherein the ion transporter activity is NH4+ ion transporter activity.

12. A fragment of the nucleic acid molecule according to claim 1 encoding a protein having an epitope of RhCG or an epitope of Rhcg.

13. The fragment of the nucleic acid molecule according to claim 12 wherein the epitope is an epitope of RhCG.

14. The fragment of the nucleic acid molecule according to claim 13 wherein the epitope of RhCG is an epitope of the C-tail (amino acids 417-479).

15. A recombinant vector comprising the nucleic acid molecule of claim 1.

16. The recombinant vector of claim 15 encoding a protein having an amino acid sequence at least 60% identical to at least a portion of SEQ ID No.: 3 or at least a portion of SEQ ID No.: 4.

17. The recombinant vector of claim 16, wherein the protein has transporter activity.

18. The recombinant vector of claim 17, wherein transporter activity is ion transporter activity.

19. The recombinant vector of claim 18, wherein the ion transporter activity is NH₄⁺ ion transporter activity.

20. A host cell transformed with the recombinant vector of claim 15.

21. The host cell of claim 20 encoding a protein having an amino acid sequence at least 60% identical to at least a portion of SEQ ID No.: 3 or at least a portion of SEQ ID No.: 4.

22. The host cell of claim 21 wherein the protein has transporter activity.

23. The host cell of claim 22 wherein transporter activity is ion transporter activity.

24. The host cell of claim 23 wherein the transporter activity is NH4⁺ ion transporter activity.

25. The host cell of claim 20 which is a mammalian host cell, an insect host cell, a fungal host cell, a yeast host cell or a bacterial host cell.

26. An isolated protein or peptide comprising an amino acid sequence at least 60% identical to at least a portion of SEQ ID No.: 3 or at least a portion of SEQ ID No.: 4.

27. The isolated protein or peptide according to claim 26 comprising an amino acid sequence encoded by the nucleic acid molecule of claim 1.

28. The isolated protein or peptide according to claim 26 comprising an epitope that is specifically bound by an antibody which specifically binds the RhCG glycoprotein or by an antibody which specifically binds the Rhcg glycoprotein.

29. The isolated protein or peptide according to claim 26 consisting of the amino acid sequence of SEQ ID No.: 3, or SEQ ID No.: 4.

30. The isolated protein or peptide according to claim 26 which is unglycosylated.

31. The isolated protein or peptide according to claim 26 which is glycosylated.

32. A fusion protein comprising a fragment of the protein or peptide of claim 26.

33. The fusion protein according to claim 32 comprising an epitope of RhCG glycoprotein or Rhcg glycoprotein.

34. The fusion protein according to claim 32 having transporter activity.

35. The fusion protein according to claim 34 having ion transporter activity.

36. The fusion protein according to claim 35 having NH₄⁺ ion transporter activity.

37. The fusion protein according to claim 32 comprising a detectable peptide.

38. The fusion protein according to claim 32 comprising a capture epitope.

39. An antibody that specifically binds to an epitope of RhCG glycoprotein or to an epitope of Rhcg glycoprotein.

40. The antibody of claim 39 wherein the antibody is a monoclonal antibody.

41. The antibody of claim 39 which is a polyclonal antibody.

42. The polyclonal antibody of claim 41 which specifically binds an epitope of RhCG glycoprotein.

43. The polyclonal antibody of claim 42 wherein the epitope of RHCG is an epitope of the N-terminal extracellular region, an extracellular loop, an intracellular loop or the C-terminal intracellular region.

44. The polyclonal antibody of claim 43 wherein the epitope is an epitope of the C-terminal intracellular region.

45. The polyclonal antibody of claim 44 wherein the epitope of the C-terminal intracellular region has an amino acid sequence within SEQ ID No.: 18.

46. A gene specific probe selected from the group consisting of SEQ ID No.: 9, SEQ ID No.: 10, SEQ ID No.: 11, SEQ ID No.: 12, SEQ ID No.: 13, SEQ ID No.: 14, SEQ ID No.: 15, SEQ ID No.: 16, SEQ ID No.: 17, SEQ ID No.: 19, SEQ ID No.: 20, SEQ ID No.: 21, SEQ ID No.: 22, SEQ ID No.: 23, SEQ ID No.: 24 and SEQ ID No.: 25.

47. An isolated nucleic acid molecule comprising a functional RhCG regulatory region or a functional Rhcg regulatory region.

48. The isolated nucleic acid according to claim 47 wherein the functional RhCG regulatory region comprises the nucleotide sequence of SEQ ID No.: 7.

49. The isolated nucleic acid according to claim 47 wherein the functional RhCG regulatory region comprises a fragment of the nucleotide sequence of SEQ ID No.: 7.

50. The isolated nucleic acid according to claim 47 wherein the functional Rhcg regulatory region comprises the nucleotide sequence of SEQ ID No.: 8.

51. The isolated nucleic acid according to claim 47 wherein the functional Rhcg regulatory region comprises a fragment of the nucleotide sequence of SEQ ID No.: 8.

52. A hybrid gene under Rh type C gene regulation comprising an upstream nucleic acid regulatory sequence of the RhCG gene and a coding sequence of a gene to be regulated.

53. The hybrid gene according to claim 52 wherein the upstream nucleic acid regulatory sequence comprises a nucleic acid sequence within the sequence of SEQ ID No.: 7.

54. The hybrid gene according to claim 52 wherein the upstream nucleic acid regulatory sequence comprises a nucleic acid sequence from SEQ ID No.: 7 up to ATG at position +1.

55. The hybrid gene according to claim 52 wherein the upstream nucleic acid regulatory sequence comprises a nucleic acid sequence from SEQ ID No.: 7 up to position −24.

56. The hybrid gene according to claim 52 wherein the upstream nucleic acid regulatory sequence comprises a nucleic acid sequence within the sequence of SEQ ID No.: 8.

57. The hybrid gene according to claim 52 wherein the upstream nucleic acid regulatory sequence comprises a nucleic acid sequence from SEQ ID No.: 8 up to ATG at position +1.

58. The hybrid gene according to claim 52 wherein the upstream nucleic acid regulatory sequence comprises a nucleic acid sequence from SEQ ID No.: 8 up to position −126.

59. A method of detecting an Rhcg or an RhCG glycoprotein in a sample, said method comprising:
(i) providing a sample to be tested,
(ii) contacting the sample with an antibody that specifically binds to an epitope of RhCG glycoprotein or an Rhcg glycoprotein under conditions suitable for binding,
(iii) assessing the specific binding to the antibody, and thereby detecting the presence of an epitope of Rhcg or RhCG glycoprotein in the sample.

60. A method of detecting an Rhcg or RhCG nucleotide sequence in a sample, said method comprising:
(i) Providing a nucleic acid sample,
(ii) contacting the sample with a nucleic acid probe that hybridizes to the nucleic acid having the sequence of SEQ ID No.: 1 or SEQ ID No.: 2, under conditions suitable for hybridization,
(iii) detecting the nucleic acid probe hybridized to the sample, and thereby detecting the presence of an Rhcg or RhCG nucleotide sequence in the sample.
